(19) United States
(12) Patent Application Publication
(10) Pub. No.: US 2026/0201046 A1
(43) Pub. Date: Jul. 16, 2026

(54) A HETEROHYBRIDOMA-BASED METHOD OF GENERATING RECOMBINANT RABBIT MONOCLONAL ANTIBODIES AND ANTIBODIES PRODUCED BY METHOD

(71) Applicant: Bio-Rad Laboratories, Inc., Hercules, CA (US)

(72) Inventors: Yu PENG, Shanghai (CN); Chune ZHANG, Shanghai (CN)

(21) Appl. No.: 19/139,172

(22) PCT Filed: Dec. 14, 2022
(86) PCT No.: PCT/CN2022/139040

§ 371 Date: Jun. 13, 2025

(51) Int. Cl. C07K 16/28; C12Q 1/6869; G01N 33/543; G01N 33/68

(52) U.S. Cl. CPC C07K 16/2866; C12Q 1/6869; G01N 33/54326; G01N 33/6857; C07K 2317/565; C12Q 2600/172; G01N 2333/7155

(57) ABSTRACT

Provided is a heterohybridoma-based method of generating recombinant rabbit monoclonal antibodies, recombinant anti-IL-6 receptor-alpha (IL-6Rα) antibodies generated using such methods, and immune assay methods kits employing such antibodies.

### REFERENCE TO A SEQUENCE LISTING SUBMITTED AS AN XML FILE

The instant application contains a Sequence Listing submitted electronically in XML format that is hereby incorporated by reference in its entirety. Said XML copy, created on Jun. 13, 2025, is named 094263-1494978_120110US_SL.xml and is 27,403 bytes in size.

### BACKGROUND

Thousands of murine mAbs against various antigens have been generated using the well-known hybridoma technology¹⁻⁴, and they are widely used in all antibody-based applications. However, the murine immune system recognizes a limited repertoire of antigens and frequently fails to respond to self-antigens. These limitations have impeded the development of murine mAbs in the past decade. Recently, it was found that the rabbit antibody repertoire was an exceptional source for generating high-sensitivity and high-specificity mAbs with diverse epitope recognition over most immunogens⁵⁻⁷. In addition, rabbits can respond to less-immunodominant epitopes, which means that the antibodies produced in rabbits usually recognize more epitopes of one target than those generated in murine animals⁸⁻¹¹. Generating rabbit mAbs therefore provides high-quality antibodies to meet the needs of fundamental research, diagnostics, and therapeutics^{7, 12-19}. As a result, rabbit mAbs have replaced murine mAbs for the detection of various important biomarkers in the pathology field, including tumor-associated antigens such as HER2, CD117, estrogen receptor, and progesterone receptor. Indeed, several rabbit mAbs manufactured by biotechnology and biopharmaceutical companies have been approved by the U.S. Food & Drug Administration (FDA) as in vitro diagnostic (IVD) products for clinical immunohistochemistry (IHC)^{20, 21}. In the therapeutic field, Novartis's Beovu (brolucizumab), a humanized vascular endothelial growth factor A (VEGFA)-targeted therapeutic antibody derived from rabbits, was approved by the FDA for the treatment of the chronic, degenerative eye disease wet age-related macular degeneration (wet AMD)²². In addition, several humanized rabbit mAb therapeutics are currently being investigated in clinical trials. These include mAb therapies and mAb-based chimeric antigen receptor T cell therapies, which have the potential to become effective therapies in the near future^{16, 17, 23-25}.

The methods used to generate rabbit mAbs can be categorized into two groups. One is based on conventional hybridoma technology, and the other is based on relatively new recombinant antibody technologies that employ molecular biology techniques, including phage display and single B cell cloning.

Among the methods in the first category, producing intact rabbit mAbs by generating rabbit-mouse heterohybridomas is a feasible practice^{26, 27}. The principle of generating heterohybridomas was also validated in other species²⁸⁻³². Subsequently, however, researchers found that those heterohybridomas always failed to secret intact antibodies after cell subcloning and prolonged culture, perhaps due to genome instability in the hybridized cells^{26, 27, 33-37}. The stable production of rabbit mAbs was finally achieved by generating cognate rabbit hybridomas employing patented rabbit plasmacytoma cell lines³⁸⁻⁴². However, the rabbit hybridomas frequently produced a low yield of antibodies, causing difficulties in antibody manufacturing^{38, 43}. In addition, studies have revealed that the conventional mAbs produced by hybridoma technology decline in purity and productivity after long-term cell culture (over months), especially in low serum and serum-free media⁴⁴⁻⁴⁶, which consequently affects their specificity and reproducibility⁴⁷⁻⁴⁹ Discussions about the worrying unreliability of a substantial portion of hybridoma-based mAb products, have drawn attention to the need to generate new types of antibodies that are truly monospecific, rigorously reproducible, and available long term⁵⁰⁻⁵³.

Phage display was originally designed to present peptides on the surface of bacteriophages to study protein-protein interactions⁵⁴. Incorporated with antibody libraries, antibody fragments containing heavy and light chain variable regions (V_{H} and V_{L}) can be presented on filamentous phages and used in the selection of mAbs in vitro⁵⁵⁻⁵⁹. Various rabbit antibody libraries have been generated from immunized rabbits and used to display antibodies as a single-chain variable fragment (scFv) and an antigen-binding fragment (Fab) for selection⁶⁰⁻⁶⁵. The scFv has a tendency to form dimers and polymers, which may cause avidity-driven selections^{58, 66}. The phage display of rabbit Fab is generally achieved using a chimeric rabbit/human Fab format, which consists of rabbit variable domains V_{H} and V_{L} fused with human constant domains CH1 and C_{L}, respectively⁶⁷⁻⁶⁹. A potential disadvantage of phage display is the compositional biases introduced during library construction and phage amplification⁷⁰⁻⁷², which reduce the diversity and selection quality.

Single B cell cloning is a molecular biology-based technique to clone the antibody gene from a single plasmablast. It starts with antigen capture and fluorescence-activated cell sorting (FACS). Using flow cytometry, antigen-specific plasmablasts are identified and isolated by capturing fluorescently conjugated antigens from animal splenocytes or animal and human peripheral blood mononuclear cells (PBMCs) following an immunization or vaccination. The genes encoding the antibodies are cloned from the single plasmablasts via single B cell molecular cloning⁷³⁻⁷⁵. In addition to FACS, cell-based microarray chip systems have been established to sort antigen-specific single B cells in order to clone antibody genes⁷⁶⁻⁷⁹. Using either approach, single B cell cloning is a rapid strategy to generate rAbs from immunized rabbits by allowing the direct sampling of the immune repertoire. Moreover, it preserves natural cognate pairing of heavy and light chains. However, despite the antigen-specific cell sorting, the success rate was low, with antigen-specific rAbs generated from only approximately 10% (or an even lower percentage) of isolated immune cells^{73, 80}. In practice, specific high-throughput facilities and FACS technique are required to ensure stable performance. Therefore, the cost and facility considerations make single B cell cloning technology preferred for developing therapeutics over other applications in fundamental research or diagnostics.

Currently, hybridoma technology, phage display, and single B cell cloning are mainstream technologies used to generate rabbit mAbs.

### BRIEF SUMMARY

In certain aspects, the disclosure provides a method of generating recombinant rabbit mAbs (rabbit rAbs) by combining cell hybridization and molecular cloning techniques, in conjunction with an approach to pair variable region heavy (H) and light (L) chains using specific parameters as described herein. In further aspects, the disclosure rAbs that bind to different epitopes of mouse soluble interleukin-6 receptor alpha from a single immunization identified using the method. Accordingly, the disclosure additionally features rAbs having affinities in the range of 10⁻⁹ to 10⁻¹² M, which exhibit superior performance over murine mAbs in a Bio-Plex application in terms of both sensitivity and dynamic range. Thus, in one aspect, the disclosure features a method of identifying a rabbit monoclonal antibody, the method comprising: (a) generating heterohybridomas comprising splenocytes obtained from a rabbit immunized with an antigen of interest fused to mouse myeloma cell; (b) screening heterohybridomas generated in (a) to identify antibodies that bind to the antigen of interest; (c) selecting heterohybridomas that express antibodies having one or more desired binding properties, e.g., binding, neutralization and the like, to provide a subpopulation for further screening; (d) obtaining V_{H} and V_{L} CDNAs from RNA isolated from each heterohybridoma identified in (b); (e) sequencing the V_{H} and V_{L} CDNA and aligning sequences to prepare a phylogenetic tree for V_{H} and V_{L} regions for each heteorhybridoma; (f) selecting a V_{H} region that comprises a CDR3 that is 8-20 residues in length; and (i) does not contain greater than 3 consecutive residues of the same amino acid; (ii) comprises a at least one aromatic residue; (iii) does not comprise three of one, or a combination of, the following amino acids: P, C, K or Q; (g) selecting a V_{L} region that comprises a CDR3 that is 8-20 residues in length; and (i) does not contain more than 3 consecutive residues of the same amino acid; (ii) comprises an aromatic amino acid; (iii) does not comprise three of one, or a combination of, the following amino acids: P, C, K or Q; (h) independently expressing a V_{H} region selected in (f) with each V_{L} region selected in (g) to provide V_{H} and V_{L} region pairs; and independently expressing each V_{L} region selected in (g) with each V_{H} region selected (f) to provide V_{H} and V_{L} pairs; and (i) analyzing the VHI and V_{L} region pairs to identify a pair that binds to the antigen of interest with the highest affinity relative to the other pairs. In some embodiments, the HCDR3 and/or the LCDR3 comprises two or more aromatic residues, wherein two aromatic residues are separated by at least one G or S residue. In some embodiments, (g) is performed before (f).

In a further aspect, the disclosure features an anti-IL-6 receptor-alpha (IL-6Rα) antibody that binds to a soluble IL-6Rα (sIL-6Rα) polypeptide, said anti-IL-6Rα antibody comprising:

(a)
HCDR1:
(SEQ ID NO: 10)
GFSFSGDYD
HCDR2:
(SEQ ID NO: 11)
TDSGFSGTT
HCDR3:
(SEQ ID NO: 12)
ARDFDSSGSYYWDL
LCDR1:
(SEQ ID NO: 13)
ESVSSNNR
LCDR2:
AAS
LCDR3:
(SEQ ID NO: 14)
AGYKGLYSDGRA;
(b)
HCDR1:
(SEQ ID NO: 15)
GFSFISYHYY
HCDR2:
(SEQ ID NO: 16)
IDAISSGST
HCDR3:
(SEQ ID NO: 17)
ARAPYYTYDGVYYALTL
LCDR1:
(SEQ ID NO: 18)
ENIYNF
LCDR2:
RAS
LCDR3:
(SEQ ID NO: 19)
QRNAYSSWA;
(c)
HCDR1:
(SEQ ID NO: 20)
GFSFSSGYY
HCDR2:
(SEQ ID NO: 21)
IYTGSDTT
HCDR3:
(SEQ ID NO: 22)
ARDLGSRGNL
LCDR1:
(SEQ ID NO: 23)
ENIYSF
LCDR2:
RAS
LCDR3:
(SEQ ID NO: 19)
QRNAYSSWA;
or
(d)
HCDR1:
(SEQ ID NO: 24)
GFSFSSNYW
HCDR2:
(SEQ ID NO: 25)
IYLFSVGNT
HCDR3:
(SEQ ID NO: 26)
ARAPYYLDGARAYYAFNL
LCDR1:
(SEQ ID NO: 27)
QSIGSD
LCDR2:
FAS
LCDR3:
(SEQ ID NO: 28)
AGGYNSADIFA.

In some embodiments, the V_{H} region has at least 95% identity to the amino acid sequence set forth in SEQ ID NO: 1 and the V_{L} region has at least 95% identity to the amino acid sequence set forth in SEQ ID NO:2; the V_{H} region has at least 95% identity to the amino acid sequence set forth in SEQ ID NO:3 and the V_{L} region has at least 95% identity to the amino acid sequence set forth in SEQ ID NO:4; the V_{H} region has at least 95% identity to the amino acid sequence set forth in SEQ ID NO:5 and the V_{L} region has at least 95% identity to the amino acid sequence set forth in SEQ ID NO:6; or the V_{H} region has at least 95% identity to the amino acid sequence set forth in SEQ ID NO:7 and the V_{L} region has at least 95% identity to the amino acid sequence set forth in SEQ ID NO:8.

In some embodiments, the antibody comprises a V_{H} region comprising the sequence set forth in SEQ ID NO:1 and a V_{L} region comprising the sequence set forth in SEQ ID NO: 2. In other embodiments, the antibody comprises a V_{H} region comprising the sequence set forth in SEQ ID NO:3 and a V_{L} region comprising the sequence set forth in SEQ ID NO: 4. In further embodiments, the antibody comprises a V_{H} region comprising the sequence set forth in SEQ ID NO:5 and a V_{L} region comprising the sequence set forth in SEQ ID NO: 6. In still other embodiments, the antibody comprises a V_{H} region comprising the sequence set forth in SEQ ID NO:7 and a V_{L} region comprising the sequence set forth in SEQ ID NO: 8.

The disclosure additionally provides kits comprising a first IL-6Rα antibody as described herein, e.g., in the preceding paragraphs coupled to a solid support. In some embodiments, such a kit further comprises a second IL-6Rα antibody comprising at least one CDR that differs in sequence from the first IL-6Rα antibody, for example, an antibody of any one of the preceding paragraphs. In some embodiments, the first antibody is coupled to a magnetic bead and the second antibody is labeled with a detectable label.

In a further aspect, the disclosure provides a method of detecting an IL-6Rα polypeptide comprising incubating a sample with a first IL-6Rα antibody as described herein, e.g., in the preceding paragraphs, wherein the antibody is labeled with a detectable label. In some embodiments, the method further comprises incubating the sample with a second IL-6Ra antibody comprising at least one CDR that differs in sequence from the first IL-6Rα antibody, wherein the antibody is coupled to a solid support, e.g., is an antibody of any one of the preceding paragraphs. In further embodiments, the method comprises detecting a signal from the detectable label.

### BRIEF DESCRIPTION OF THE DRAWINGS

FIG. 1. The principle of Bio-Plex assays. Bio-Plex assays are immunoassays essentially formatted on magnetic beads and fluorescent reporters. In the assays, capture antibodies which are directed against the biomarker of interest are covalently coupled to the beads. The coupled bead binds to the biomarker in test sample. After several washes to remove unbound protein, a biotinylated detection antibody is added and binds to the biomarker to create a sandwich complex. The final detection complex is formed after the addition of streptavidin-phycoerythrin conjugates. Phycoerythrin serves as the fluorescent reporter.

FIG. 2A-D provides a schematic of rabbit immunization and generation of rabbit-mouse heterohybridomas. A) A full length mouse IL-6Ra protein consists of a secretion signal peptide (Met¹-Ala¹⁹, green box, SS), an extracellular domain (Leu²⁰-Pro³⁶⁴, blue box, ED), a single transmembrane helical domain (Thr³⁶⁵-Leu³⁸⁵, orange box, TM) and a cytoplasmic domain (Arg³⁸⁶-Arg⁴⁶⁰, purple box, CD). The proteolytic cleavage site is indicated by an arrow in black (Gln³⁵⁷/Glu³⁵⁸). The soluble form of mouse IL-6Ra includes the residues of Leu²⁰-Gln³⁵⁷. With an addition of the N-terminal polyhistidine affinity tag (His₁₀-tag, red box) (“His₁₀” disclosed as SEQ ID NO: 29), a recombinant soluble IL-6Ra was produced for rabbit immunization. B) A schematic presents the schedule of immunizing rabbits and isolating splenocytes. 4 subcutaneous injections were given in each week at indicated doses (blue arrow). C) A workflow presents the steps of generating recombinant rabbit monoclonal antibodies from heterohybridomas. The rabbit-mouse heterohybridomas were generated by fusing rabbit splenocytes with mouse Sp2/0 cells. After screening, the rabbit antibody genes were isolated from the positive fusion clones and subjected to bioinformatics analysis. A prior pool containing selected antibody genes was established for each clone. Recombinant antibodies encoded by these genes were screened in Bio-Plex application to identify target-specific ones. D) A schematic describes the stages of screening rabbit-mouse heterohybridoma fusion clones.

FIG. 3A-C depicts identification of rabbit rAbs. A) A simplified work flow of selecting the sequence to establish a prior pool for each fusion clone. B) The primary pool of each clone consists of productive genes encoding H chains and L chains. The prior pool of each clone contains the gene sequences selected from the corresponding primary pool. The number of H-L pairs is a product of the number of H chain and that of L chain in each pool. C) The activity of rabbit rAbs which were encoded by the H-L pairs in prior pools were measured as detection antibodies in Bio-Plex application. The activity against the recombinant mouse sIL-6Ra at 32000 pg/mL (signal, S) or 0 pg/mL (background, B) was evaluated by a S/B ratio. Each dot in the scatter plot represents the S/B ratio of a H-L pair. C, Left to right: Clone 322, Clone #48, Clone #52, Clone #62,

FIG. 4A-E provide data illustrating the performance of selected antibodies in Bio-Plex a assay. A) The performance of rabbit rAbs as detection and capture antibodies in Bio-Plex application was evaluated by a S/B ratio when detecting the recombinant mouse sIL-6Ra at 32000 pg/mL (signal, S) and 0 pg/mL (background, B). B) The sensitivity of rabbit rAbs (except rAb #48) were compared with that of an in-house rat monoclonal antibody in detecting recombinant mouse sIL-6Ra at serial dilutions. C) The purified rabbit rAbs were tested in pairwise. The activity of C-D pairs were evaluated by a S/B ratio when detecting the recombinant mouse sIL-6Ra at 32000 pg/mL (signal, S) and 0 pg/mL (background, B). The numbers in red suggest unexpected low values of S/B ratio. D) A schematic shows the mutual compatibility of three rAbs as C-D pairs. E) The columns in the plot describe the linear range of detection when using indicated pairs of antibodies to detect the recombinant mouse sIL-6Ra in a series of dilutions. The numbers at the top and the bottom of columns are the upper and lower limit of quantitation, respectively. A-C, E, Standard points and samples were assayed in duplicate.

FIG. 5A-I. The binding of antibodies to mouse sIL-6Ra in vitro.A) Using structure-based modeling, the antigen-antibody interaction was predicted in an in silico simulation. The rabbit rAb models (spacefilling diagram, VH is in red and VL is in cyan) and the model of mouse sIL-6Ra dimer (ribbon diagram, in red) were illustrated in backbone diagrams. B) Antigen-antibody binding affinity of the rabbit rAbs and the rat monoclonal antibody were determined using SPR. C) The principle of a competitive assay is illustrated. The antigen is immobilized to a surface and antibodies are added tandemly into the reaction. Once the binding of the competitor antibody (blue) with the antigen blocks the subsequent binding of a test antibody (pink), a drop of response unit (RU) occurs (red arrow). D-I) In each plot, the results of three tandem assays in one channel were merging to exhibit the drop of response unit (RU). The curves represent the antigen-antibody binding of test antibody (red), competitor antibody (blue) and competitive binding of the test antibody to the recombinant mouse sIL-6Rα at the presence of competitor antibody (cyan). The drop of response in competitive binding was indicated by red arrow in H and I. There is no significant drop of response in D-G.

### TERMINOLOGY

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein refers to the usual error range for the respective value readily known to the skilled person in this technical field. For example, for K_{D} and IC₅₀ values±20%, ±10%, or ±5%, are within the intended meaning of the recited value.

The term “interleukin-6 Receptor alpha” or “IL-6Rα” as used here refers to a the polypeptide that is encoded by an IL6R gene. The human IL6r gene is cytogenetically localized to human chromosome 1q21.3. An illustrative human soluble IL-6Rα (sIL6R) protein sequence is available under Uniprot number P08887-2 and is provided as SEQ ID NO: 9. A human IL-6R cDNA sequence is available under NCBI accession number NM_000565, e.g., NM_000565.4. As used herein a “soluble IL-6Rα” refers to any soluble IL-6Rα polypeptide encoded by an IL-6R gene. Orthologs from other species are well known, including cow, horse, macaque, chimpanzee, dog, cat, mouse, and rat (see, for example HGNC symbol report for IL-6R and resources listed therein.).

The terms “anti-IL-6Rα antibody,” “IL-6Rα specific antibody,” “IL-6Rα antibody,” and “anti-IL-6Rα” are used synonymously herein to refer to an antibody as described herein that specifically binds to IL-6Rα. An illustrative human sIL-6Rα sequence is provided in SEQ ID NO:9. In illustrative embodiments described herein, an anti-IL-6Rα antibody can also bind to other species, e.g., mouse, rat, dog, cat, and the like.

An “anti-IL-6Rα binding domain” as used herein refers to an antigen binding domain comprising a V_{H} and a V_{L} region of an anti-IL-6Rα antibody as described herein.

The term “antibody” refers to a polypeptide comprising a framework region encoded by an immunoglobulin gene, or fragments thereof, that specifically binds and recognizes an antigen, e.g., IL-6. The “variable region” contains the antigen-binding region of the antibody (or its functional equivalent) and is important in specificity and affinity of binding. The term “antibody” as used herein thus encompasses antigen binding fragments, e.g., an antigen binding domain, or other antigen binding fragment. Antigen binding fragments may be produced by modification of whole antibodies, or produced using recombinant DNA methodologies (e.g., single chain Fv formats).

An illustrative immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

As used herein, “V-region” refers to an antibody, e.g., antibody, variable region domain comprising the segments of Framework 1, CDR1, Framework 2, CDR2, and Framework 3, including CDR3 and Framework 4, which segments are added to the V-segment as a consequence of rearrangement of V-region genes during B-cell differentiation.

As used herein, “complementarity-determining region (CDR)” refers to the three hypervariable regions that interrupt the four “framework” regions of a variable domain. The CDRs are the primary contributors to binding to an epitope of an antigen. The CDRs of are referred to as CDR1, CDR2, and CDR3, numbered sequentially starting from the N-terminus.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M.J.E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

An “isotype” is a class of antibodies defined by the heavy chain constant region. Antibodies described herein can be of any isotype of isotype class. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively. In some embodiments, the IgG is an IgG1, IgG2, IgG3 or IgG4.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F (ab)′₂, a dimer of Fab which itself is a light chain joined to V_{H}—CH1 by a disulfide bond. The F (ab)′₂ may be reduced under mild conditions to break the disulfide linkage in the hinge region, thereby converting the F (ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies. The term “antibody” additionally encompasses bispecific and multispecific antibodies as well as any other monovalent, bivalent, or multivalent antibody format.

The various antibodies or antigen-binding fragments described herein can be produced by enzymatic or chemical modification of the intact antibodies, or synthesized de novo using recombinant DNA methodologies (e.g., single chain Fv), or identified using yeast or phage display libraries (see, e.g., McCafferty et al., Nature 348:552-554, 1990; Boder, et al (2000) Proc. Natl. Acad. Sci. U.S.A 97:10701) constructed from synthetic, semi-synthetic or naive or immunocompetent sources.

A “monoclonal antibody” refers to a clonal preparation of antibodies with a single binding specificity and affinity for a given epitope on an antigen.

As used herein, the term “specifically binds to,” as used with reference to an affinity agent, refers to an affinity agent (e.g., an antibody) that binds to an antigen with at least 5-fold greater affinity than to non-IL-6Rα antigen molecule, e.g., 6-fold, 7-fold, 8-fold, 9-fold, 10-fold, 20-fold, 25-fold, 50-fold, 100-fold, 10³-fold, 10⁴-fold, 10⁵-fold, 10⁶-fold, 10⁷-fold, 10⁸-fold, 10⁹-fold, 10¹⁰-fold, 10¹¹-fold, 10¹²-fold, 10¹³-fold, 10¹⁴-fold, or 10¹⁵-fold greater affinity.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “conservative” substitution as used herein with respect to a protein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. Nucleic acid molecules, e.g. oligonucleotide probes or priomers, may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes sequences comprising a degenerate codon, e.g., codon-optimized nucleic acids, that encode the same polypeptide sequence.

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody paratope, or a sequence that is at least about 25 amino acids or nucleotides in length, or over a region that is 50-100 amino acids or nucleotides in length, or over the entire length of the reference sequence.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a variable domain polypeptide “corresponds to” an amino acid in the variable domain polypeptide of SEQ ID NO: 1 when the residue aligns with the amino acid in SEQ ID NO: 1 when optimally aligned to SEQ ID NO: 1. The polypeptide that is aligned to the reference sequence need not be the same length as the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

### Method of Generating Recombinant Rabbit Monoclonal Antibodies

In one aspect, the present invention provides a method of generating recombinant rabbit monoclonal antibodies using heterohybridomas. In brief, rabbits are immunized with an antigen to which antibodies are desired. Following immunization, splenocytes are isolated and fused with mouse hybridoma cells, e.g., Sp2/0 cells, using known techniques (see, e.g., detailed technical section below. Fusion clones are then screened, e.g., via ELISA to identify supernatants that exhibit high activity against the antigen. The top performing clones can then be identified.

Genes encoding VH and VL region are then isolated. A cluster of several different productive V_{H} and V_{L} sequences are typically identified from each fusion clone. These genes comprised dozens of combinations of V_{H} and V_{L} pairs (H-L pairs). To simplify screening to identify functional recombinant antibodies, a sequence-based approach is employed to select high-priority sequences from the clusters and establish prior pools from which to identify the functional recombinant antibodies. This approach, called Sequence Priority by Residual Preference (SPRP), leverages universal features of residue preference in the antigen-binding region of functional antibodies⁹⁷⁻¹⁰¹. For selection, antibody genes in which the CDR3 is of moderate length (8-20 residues) are generally preferred. Further CDR3s containing unusual fragments, such as tandem repeats of serine residues, and unfavorable residues are excluded. This results in a reduction in the number of H-L pairs for pairwise testing.

In typical selection protocols, sequence alignments of the amino acid sequences of VH regions and alignment sof the amino acid sequences of VL regions are performed, e.g., using a program such as MUSCLE (ebi.ac.uk/Tools/msa/muscle/) to generate phylogenetic trees. Based on the alignments and trees, the following criteria are employed for selection of VH regions and VL regions:
a) V regions are selected that have CDR3s possessing moderate length (numbering IMGT) of 8-20 residues.
b) V regions are selected that have a CDR3 those that does not contain greater than 3 consecutive residues of the same amino acid;
c) V regions are selected that do not comprise three of one, or a combination of, the following amino acids: P, C, K or Q; and optionally, comprises at least one aromatic residue, e.g. Y; In some embodiments, a sequence having an HCDR3 and/or a sequence having an LCDR3 is selected that comprises two or more aromatic residues, wherein two aromatic residues are separated by at least one G or S residue.
   Combinations of selected VH and VL regions can then be generated by expressing V_{H} and V_{L} region pairs.

Plasmids encoding the antibody genes defined above are introduced into mammalian cells pairwise by transient transfection. Their corresponding rAbs are produced and secreted into the culture medium. In some embodiments, e.g., for developing antibodies for Bio-Plex assay, the cell culture supernatants are assessed for activity using a Bio-Plex to assess their activity as a detection antibody. In the screening, the rAb of supernatants that possessed the highest activity are identified as the functional rAb produced by its original fusion clone.

Anti-IL-6Rα Antibodies from Rabbit Heterohybridomas

Provided herein are anti-IL-6Rα antibodies that bind to soluble IL-6Rα (sIL-6Rα), e.g., that can be used for detecting sIL-6Rα from various species, including human sIL-6Rα.

In some embodiments, an anti-IL-6Rα antibody of the present disclosure has an affinity ranging from 10⁻⁹ to 10⁻¹² M.

In some embodiments, an anti-IL-6Rα binding domain of the present disclosure has at least one, at least two, or three CDRs of a variable domain sequence of a heavy chain variable domain of SEQ ID NO: 1 or a light chain variable domains of SEQ ID NO:2. In some embodiments, an anti-IL-6Rα binding domain of the present disclosure comprises an HCDR3 of SEQ ID NO:1 and an LCDR3 of SEQ ID NO:2. In some embodiments, an anti-anti-IL-6Rα binding domain comprises an HCDR1, HCDR2, and HCDR3 of SEQ ID NO:1 and LCDR1, LCDR2, and LCDR3 of SEQ ID NO:2.

In some embodiments, an anti-IL-6Rα binding domain comprises an HCDR1, HCDR2, and HCDR3 in which one of the CDRs, or two of the CDRs, comprise a one or two amino acid substitution, e.g., a conservative substitution, relative to the corresponding CDR set forth in SEQ ID NO:1. In some embodiments, an anti-IL-6Rα binding domain comprises an LCDR1, LCDR2, and LCDR3 in which one of the CDRs, or two of the CDRs, comprises a one or two amino acid substitution, relative to the corresponding CDR set forth in SEQ ID NO: 2.

In some embodiments, an anti-IL-6Rα binding domain comprises an HCDR1, HCDR2, and HCDR3 in which one of the CDRs, or two of the CDRs, comprise a one or two amino acid substitution, e.g., a conservative substitution, relative to the corresponding CDR set forth in SEQ ID NO:3. In some embodiments, an anti-IL-6Rα binding domain comprises an LCDR1, LCDR2, and LCDR3 in which one of the CDRs, or two of the CDRs, comprises a one or two amino acid substitution, relative to the corresponding CDR set forth in SEQ ID NO: 4.

In some embodiments, an anti-IL-6Rα binding domain comprises an HCDR1, HCDR2, and HCDR3 in which one of the CDRs, or two of the CDRs, comprise a one or two amino acid substitution, e.g., a conservative substitution, relative to the corresponding CDR set forth in SEQ ID NO:5. In some embodiments, an IL-6Rα binding domain comprises an LCDR1, LCDR2, and LCDR3 in which one of the CDRs, or two of the CDRs, comprises a one or two amino acid substitution, relative to the corresponding CDR set forth in SEQ ID NO: 6.

In some embodiments, an anti-IL-6Rα binding domain comprises an HCDR1, HCDR2, and HCDR3 in which one of the CDRs, or two of the CDRs, comprise a one or two amino acid substitution, e.g., a conservative substitution, relative to the corresponding CDR set forth in SEQ ID NO:7. In some embodiments, an IL-6Rα binding domain comprises an LCDR1, LCDR2, and LCDR3 in which one of the CDRs, or two of the CDRs, comprises a one or two amino acid substitution, relative to the corresponding CDR set forth in SEQ ID NO: 8.

In some embodiments, an anti-IL-6Rα binding domain of the present disclosure comprises a heavy chain variable region having the HCDR1, HCDR2, and HCDR3 of SEQ ID NO: 1 and wherein the variable region has at least 75%, 80%, 85%, 90%, 91%, 92%, 93%, 94%, 95%, 96%, 97%, 98%, or 99% sequence identity to the amino acid sequence of heavy chain variable region sequence of SEQ ID NO:1; and a light chain variable region having the LCDR1, LCDR2, and LCDR3 of SEQ ID NO:2 and wherein the light chain variable region hast least 75%, 80%, 85%, 90%, 91%, 92%, 93%, 94%, 95%, 96%, 97%, 98%, or 99% sequence identity to the amino acid sequence of light chain variable region sequence of SEQ ID NO: 2. In some embodiments, the variable domain comprises substitutions, insertions, or deletions in the framework of a heavy chain variable region as shown in SEQ ID NO:1. In some embodiments, the variable domain comprises substitutions, insertions, or deletions in the framework of a light chain variable region as shown in SEQ ID NO:2.

In some embodiments, an anti-IL-6Rα binding domain of the present disclosure comprises a heavy chain variable region having the HCDR1, HCDR2, and HCDR3 of SEQ ID NO: 3 and wherein the variable region has at least 75%, 80%, 85%, 90%, 91%, 92%, 93%, 94%, 95%, 96%, 97%, 98%, or 99% sequence identity to the amino acid sequence of heavy chain variable region sequence of SEQ ID NO:3; and a light chain variable region having the LCDR1, LCDR2, and LCDR3 of SEQ ID NO:4 and wherein the light chain variable region hast least 75%, 80%, 85%, 90%, 91%, 92%, 93%, 94%, 95%, 96%, 97%, 98%, or 99% sequence identity to the amino acid sequence of light chain variable region sequence of SEQ ID NO: 4. In some embodiments, the variable domain comprises substitutions, insertions, or deletions in the framework of a heavy chain variable region as shown in SEQ ID NO:3. In some embodiments, the variable domain comprises substitutions, insertions, or deletions in the framework of a light chain variable region as shown in SEQ ID NO:4.

In some embodiments, an anti-IL-6Rα binding domain of the present disclosure comprises a heavy chain variable region having the HCDR1, HCDR2, and HCDR3 of SEQ ID NO: 5 and wherein the variable region has at least 75%, 80%, 85%, 90%, 91%, 92%, 93%, 94%, 95%, 96%, 97%, 98%, or 99% sequence identity to the amino acid sequence of heavy chain variable region sequence of SEQ ID NO:5; and a light chain variable region having the LCDR1, LCDR2, and LCDR3 of SEQ ID NO:6 and wherein the light chain variable region hast least 75%, 80%, 85%, 90%, 91%, 92%, 93%, 94%, 95%, 96%, 97%, 98%, or 99% sequence identity to the amino acid sequence of light chain variable region sequence of SEQ ID NO: 6. In some embodiments, the variable domain comprises substitutions, insertions, or deletions in the framework of a heavy chain variable region as shown in SEQ ID NO:5. In some embodiments, the variable domain comprises substitutions, insertions, or deletions in the framework of a light chain variable region as shown in SEQ ID NO:6.

In some embodiments, an anti-IL-6Rα binding domain of the present disclosure comprises a heavy chain variable region having the HCDR1, HCDR2, and HCDR3 of SEQ ID NO: 7 and wherein the variable region has at least 75%, 80%, 85%, 90%, 91%, 92%, 93%, 94%, 95%, 96%, 97%, 98%, or 99% sequence identity to the amino acid sequence of heavy chain variable region sequence of SEQ ID NO:7; and a light chain variable region having the LCDR1, LCDR2, and LCDR3 of SEQ ID NO:8 and wherein the light chain variable region hast least 75%, 80%, 85%, 90%, 91%, 92%, 93%, 94%, 95%, 96%, 97%, 98%, or 99% sequence identity to the amino acid sequence of light chain variable region sequence of SEQ ID NO: 8. In some embodiments, the variable domain comprises substitutions, insertions, or deletions in the framework of a heavy chain variable region as shown in SEQ ID NO:7. In some embodiments, the variable domain comprises substitutions, insertions, or deletions in the framework of a light chain variable region as shown in SEQ ID NO:8.

In some embodiments, an anti-IL-6Rα antibody of the present disclosure, or an antigen binding domain comprising an anti-IL-6Rα antibody of the present disclosure, may be linked to detectable label. The terms “label” and “detectable label” interchangeably refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which may be made detectable, e.g., by incorporating a label into an oligonucleotide, peptide, or antibody specifically reactive with a target molecule. The term includes combinations of single labeling agents, e.g., a combination of fluorophores that provides a unique detectable signature, e.g., at a particular wavelength or combination of wavelengths. A “detectable label” as used herein includes reference to an oligonucleotide, which can be detected by PCR, sequencing or other biochemical reactions.

A molecule that is “linked” to a label (e.g., a labeled antibody) is one that is bound, either covalently, through a linker or a chemical bond, or noncovalently, through ionic, van der Waals, electrostatic, or hydrogen bonds to a label such that the presence of the molecule may be detected by detecting the presence of the bound label.

In some embodiments, an antibody of the present disclosure is immobilized on a solid support surface. Such surfaces include, but are not limited to, a bead, a microsphere, a polymer matrix, a channel, a nanoparticle, or any other surface. In typical embodiments, an antibody used in a bio-plex assay is immobilized onto the surface of a bead or microparticle.

A “solid support” refers to a material or group of materials having a rigid or semi-rigid surface or surfaces. In some embodiments, the solid support is matrix, such as a hydrophilic polymer, e.g., a polymer that is insoluble and swells in water. Suitable polymers include, but are not limited to polyhydroxy polymers, e.g., based on polysaccharides, such as agarose, dextran, cellulose, starch, pullulan, etc. and completely synthetic polymers, such as polyacrylic amide, polymethacrylic amide, poly(N,N-dimethylacrylamide, poly(hydroxyalkylvinyl ethers), poly(hydroxyalkylacrylates) and polymethacrylates (e.g. polyglycidylmethacrylate), polyethylene glycol polymers, polyvinyl alcohols and polymers based on styrenes and divinylbenzenes, and copolymers in which two or more of the monomers corresponding to the above-mentioned polymers are included. Polymers, which are soluble in water, may be derivatized to become insoluble, e.g. by cross-linking and by coupling to an insoluble body via adsorption or covalent binding. Hydrophilic groups can be introduced on hydrophobic polymers (e.g. on copolymers of monovinyl and divinylbenzenes) by polymerization of monomers exhibiting groups which can be converted to OH, or by hydrophilization of the final polymer, e.g. by adsorption of suitable compounds, such as hydrophilic polymers. In some embodiments, the support is an UNOsphere™ support, a polymer produced from water-soluble hydrophilic monomers (Bio-Rad, Hercules, CA). Alternatively, the matrix is agarose (GE Sepharose or Sterogene Superflow and Ultraflow). In some embodiments, the support is comprised of glass or synthetic fibers that have been activated with chemical groups that react with protein or nucleic acids such as Fabs or aptamers with fast off-rates. In still other embodiments, the support is a membrane material, such as nitrocellulose that can non-covalently bind the capture agents having fast off-rates as described in the section detailing lateral flow assay embodiments of the invention.

Many different techniques can be used to immobilize capture agents to a solid support. In some embodiments, immobilized carboxylate groups on an amine-reactive surface can be used to covalently link an antibody to the substrate via an amine-coupling reaction. Other illustrative reactive linking groups, e.g., hydrazines, hydroxylamines, thiols, carboxylic acids, epoxides, trialkoxysilanes, dialkoxysilanes, and chlorosilanes, may be attached to the substrate, such that polypeptides can form chemical bonds with those linking groups to immobilize them on the substrate. In some embodiments the antibody can be copolymerized into the solid support by first adding the appropriate chemical group such as an acrylic group to the antibody. In some embodiments the antibody can be attached using click chemistry. In some embodiments the capture agents are bound non-covalently to a support, such as to nitrocellulose membranes, following deposition of the agents onto the support.

In some instances, an antibody can be immobilized on a surface using methods such as a hydrophilic self-assembled monolayer approach, a hydrophilic polymer brush approach, a zwitterionic polymer brush approach and a nitrile coating approach.

In some embodiments, the surface may also be coated with streptavidin at a known concentration, followed by attachment of biotin labeled antibody.

### Technical Aspects

This section provides technical guidance for a method to generate rabbit rAbs by combining the cell hybridization and conventional molecular biology techniques, including mRNA isolation, reverse transcription polymerase chain reaction (RT-PCR) and molecular cloning. Furthermore, this section describes a sequence-based approach, termed Sequence Priority by Residual Preference (SPRP), to select high-priority antibody sequences for testing their activity. Such antibodies have many therapeutic use and uses as detection agents to detect a compound.

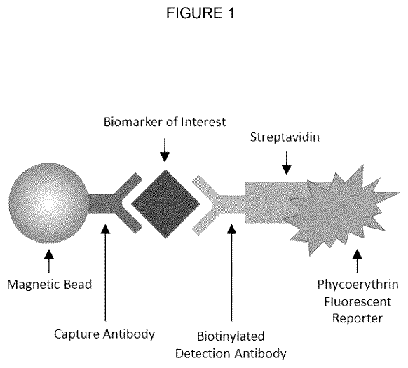

In one embodiments, such antibodies can be used in Bio-Plex assays, which are based on the principles of a sandwich enzyme-linked immunosorbent assay (ELISA). Bio-Plex involves two different antibodies binding to one target simultaneously. These antibodies are termed the “capture antibody” and the “detection antibody” (FIG. 1).

In this section, for illustration and more detailed explanation of techniques, the target is interleukin 6 receptor alpha (IL-6Ra), a component of the receptor for the pleiotropic cytokine interleukin 6 (IL-6), which is highly conserved across various species. IL-6Ra exerts its signal via two routes. In classic signaling, IL-6Rα interacts with a receptor complex composed of IL-6Rα and a second receptor subunit, glycoprotein 130 (gp130), to trigger intracellular signaling⁸¹. Alternatively, IL-6Rα can engage a soluble form of IL-6R called sIL-6Rα, to form a complex that signals through the gp130 dimer without requiring membrane-anchored IL-6Rα. This signaling pathway is known as IL-6Rα trans-signaling⁸². sIL-6Rα can be generated by either alternative RNA splicing or proteolytic cleavage by metalloproteinases⁸³⁻⁸⁵. Findings have revealed that sIL-6Ra plays a central role in several inflammatory and autoimmune diseases and cancers⁸⁶⁻⁹⁰, which makes sIL-6Rα a valuable biomarker and a potential therapeutic target in biomedical research⁹¹. Using antibody-based applications to assay sIL-6Rα in fluid samples is a direct and efficient approach to measure its fluctuations in patients and disease models 92-94. Although a handful of pAbs can detect SIL-6Rα, sIL-6Rα-targeted mAbs are rare and sIL-6Rα-specific rabbit rAbs are unavailable on the market. Therefore, generating rabbit rAbs against sIL-6Rα fills this gap and provides high-quality products to researchers.

A method to generate functional rabbit rAbs from a single immunization and to pair rabbit rAbs for use as capture and detection antibodies (C-D antibody pair) is described below. This method offers a convenient and cost-effective approach for academic and industrial investigators to customize rabbit rAbs for their specific research and applications.

### Animal Immunization and Generation of Rabbit-Mouse Heterohybridomas

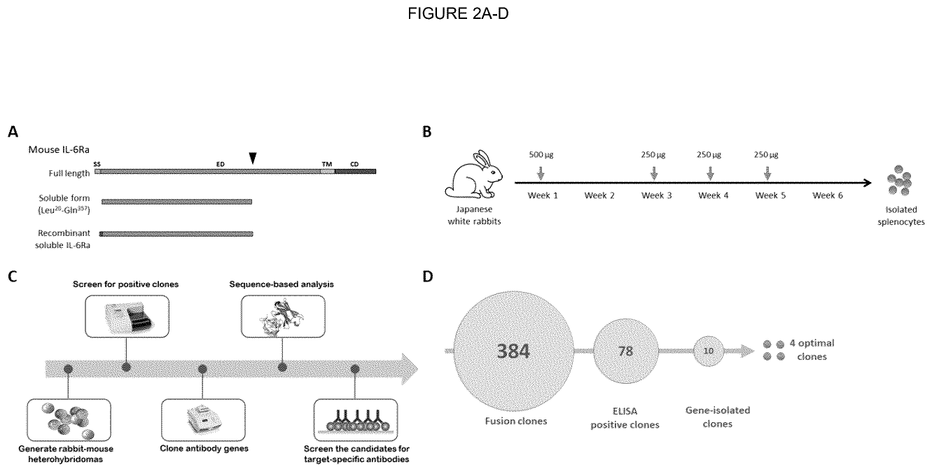

Mouse sIL-6Ra is an extracellular segment of full-length IL-6Ra that was shed from its membrane anchor after proteolytic cleavage by the metalloproteinases ADAM10 and ADAM17⁸³⁻⁸⁵. The findings that recombinant mouse sIL-6Ra is bioactive in vitro and in vivo suggest that it is an ideal immunogen for generating antibodies against its naïve protein^{95, 96} (FIG. 2A). In this study, recombinant sIL-6Ra was expressed in mammalian cells and purified using polyhistidine-tag affinity chromatography (Materials and Methods). The purified protein was mixed with adjuvants as an immunogen to immunize two rabbits (FIG. 2B). After immunization, the rabbit with the higher serum titer was sacrificed. Its splenocytes were isolated and fused with mouse Sp2/0 cells to generate heterohybridomas (FIG. 2C). All of the heterohybridoma cells were dispensed evenly into the wells of four 96-well microplates. Unfused cells were selected against using media containing HAT, and all of the remaining cells in a well were counted as one fusion clone. The fusion clones continued to secrete rabbit antibodies into their supernatants during a 7-day cell culture. Next, all 384 fusion clones from the 4 microplates were screened via ELISA and subsequent Bio-Plex application by examining the activity of the antibodies in their supernatants. After screening, 78 fusion clones whose cell culture supernatants exhibited high activity against recombinant mouse sIL-6Ra in the Bio-Plex application were selected. Among them, the top 10 clones (based on the antibody activity of their supernatants) were subjected to gene isolation, and 4 of them (clones #22, #48, #52, and #62) gave quality cDNA after reverse transcription (FIG. 2D).

### Generation of Recombinant Rabbit Monoclonal Antibodies

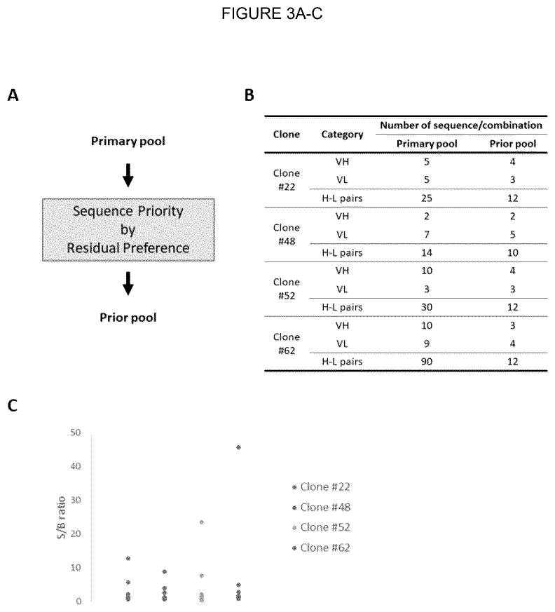

The genes encoding the V_{H} and V_{L} of rabbit antibodies were isolated from the fusion clones #22, #48, #52, and #62 (Materials and Methods). A cluster of several different productive V_{H} and V_{L} sequences were identified from each fusion clone. These genes comprised dozens of combinations of V_{H} and V_{L} pairs (H-L pairs). Screening all of these combinations for those encoding a functional rAb is a comprehensive but cost-inefficient solution. To simplify this process, we developed a sequence-based approach to select high-priority sequences from the clusters and establish prior pools from which to identify the functional rAb (Materials and Methods, FIG. 3A). This approach, called Sequence Priority by Residual Preference (SPRP), was developed based on universal features of residue preference in the antigen-binding region of functional antibodies⁹⁷⁻¹⁰¹. In the antigen-binding region, the third complementarity determining regions (CDR3s) are well known for their high variance in amino acid residues and critical roles in determining the antigen-binding specificities of antibodies¹⁰². The principle of SPRP is the meticulous interrogation of the properties of the residues in the CDR3 based on antibody sequences and the selection of those that meet the criteria. To this end, the antibody genes in which the CDR3 was of moderate length (8-20 residues) were preferred. In addition, CDR3s containing unusual fragments, such as tandem repeats of serine residues, and unfavorable residues are excluded. (The details of SPRP are described in the Materials and Methods.) Accordingly, the number of H-L pairs for the pairwise test was reduced to 10-12 after establishing the prior pools (FIG. 3B). Plasmids encoding the antibody genes defined in prior pools were introduced into mammalian cells pairwise by transient transfection. Their corresponding rAbs were produced and secreted into the culture medium. In a quick screening, the cell culture supernatants were examined using Bio-Plex to assess their activity as a detection antibody. In the screening, the rAb whose supernatant possessed the highest activity was identified as the functional rAb produced by its original fusion clone (FIG. 3C). Hence, by employing the cell hybridization technique and molecular biology methods, 4 rabbit rAbs (rAb #22, rAb #48, rAb #52, and rAb #62) were generated successfully.

### Test of Recombinant Rabbit Monoclonal Antibody Pairs in Bio-Plex

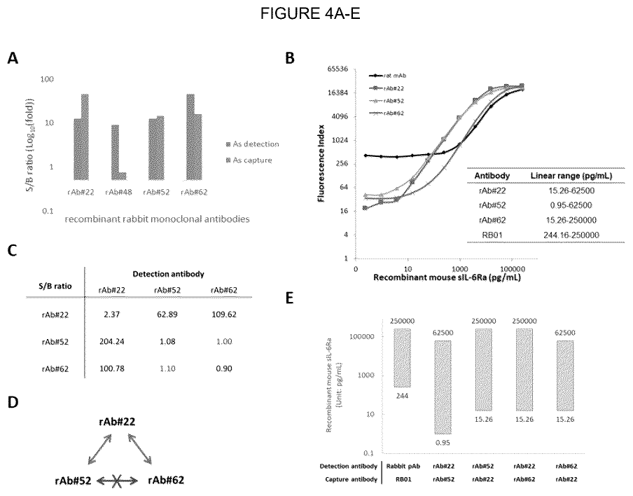

The performance of the 4 rabbit rAbs in detecting recombinant mouse sIL-6Rα, using the rAbs as both the detection antibody and the capture antibody, was examined. We found that 3 of them (rAb #22, rAb #52, and rAb #62) exhibited satisfying signal/background ratio as both the capture and the detection antibody (FIG. 4A). Their quality as the capture antibody was determined and compared with a rat mAb (RB01) that was developed in house. In the assay, these 3 rAbs were superior to the rat mAb RB01 in both background and sensitivity (FIG. 4B). Subsequently, their compatibility as C-D antibody pairs were examined in the assay. The results revealed that rAb #22 was compatible with rAb #52 and rAb #62, whereas rAb #52 and rAb #62 were mutually incompatible at both the capture and detection sides (FIG. 4C, FIG. 4D). This result suggests that rAb #22 recognizes an epitope that is different than that detected by the other two rAbs (rAb #52 and rAb #62). When the compatible rAb pairs were used to quantify serially diluted recombinant sIL-6Rα, the performance of two C-D rAb pairs (rAb #22/rAb #52 and rAb #22/rAb #62) were superior to that of a reference C-D antibody pair composed of the rat mAb RB01 and a rabbit pAb: The rAb pairs exhibited reduced background and an extended dynamic range (FIG. 4E). Moreover, when detecting native sIL-6Rα in mouse serum samples, the rabbit rAb pairs were 16-fold more sensitive than the reference pair (data not shown).

### Affinity Determination and Competitive Analysis Using Surface Plasmon Resonance

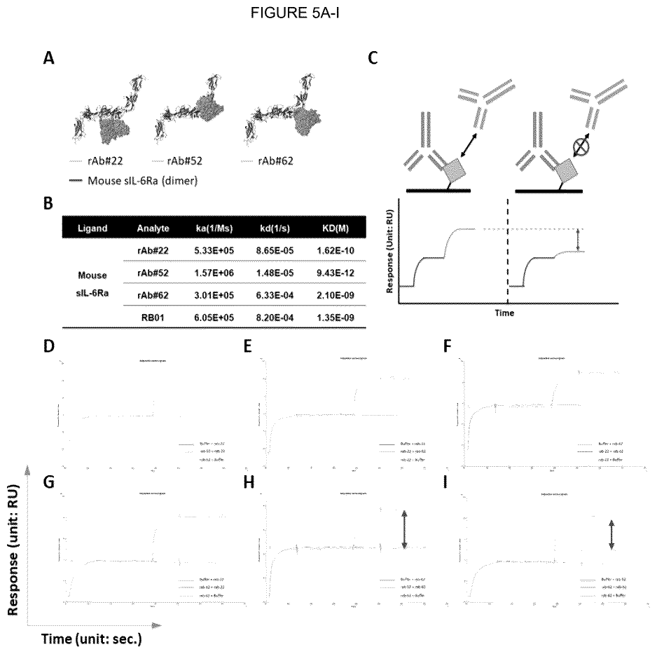

Steric hindrance is a major cause of failure in antibody pairing in Bio-Plex and other sandwich-like applications. We presumed that the incompatibility of rAb #52 and rAb #62 could also be a result of steric hindrance. In an in silico simulation, the rAb structure models were docked to a mouse sIL-6Ra structure model to predict their interactions. In this simulation, rAb #52 and rAb #62 bound to adjacent sites on sIL-6Ra, which were distant from the site to which rAb #22 bound (FIG. 5A). This result suggests that rAb #52 and rAb #62 may spatially obstruct each other during the antigen-antibody interaction. To test this hypothesis, the antigen-antibody affinities between rAb #22, rAb #52, and rAb #62 and recombinant sIL-6Ra were measured by surface plasmon resonance (SPR) (FIG. 5B). Among the rAbs, rAb #52, which exhibited the highest sensitivity in assays (0.95 μg/mL antigen), also possessed the highest affinity for recombinant sIL-6Ra (KD=9.43 PM). Similarly, the affinity of the other two rAbs was highly consistent with their sensitivity in the Bio-Plex application (FIG. 4B, FIG. 5B). Next, the SPR method was used to study the competition between these rAbs when binding to sIL-6Ra. For the competitive assay, two antibodies at the same concentration (200 nM) were injected into a sensor chip coated with sIL-6Ra antigen in order. Their binding to the antigen were evaluated by visualizing the resonance unit (RU) shift in the sensorgrams. In this assay, competition between two antibodies were identified by a reduction in RUs (FIG. 5C). Using this method, a competitive blocking profile was generated for each antibody relative to the other, which showed tandem blocking between rAb #52 and rAb #62 (FIG. 5D-I). This result suggests that, consistent with the computational simulation, these two antibodies spatially interfere with each other in the antigen-antibody interaction, which causes their incompatibility in Bio-Plex assays.

Conventional hybridoma-derived monoclonal antibodies have been a pillar of antibody-based applications for decades. Although their relevance in research and diagnostics is steadily growing, concerns about reproducibility are also rising^{48, 50}. In order to address the issues, different technologies have been employed to generate rAbs from various species for a number of applications.

In order to generate rabbit rAbs for Bio-Plex, we developed a method incorporating cell hybridization and molecular biology techniques aided with an antibody H-L chain pairwise approach SPRP. How to pair antibody heavy and light chains is a frequently asked question in rAb generation. In spite of tremendous achievements in the computational simulation of protein structure from sequences¹⁰³, it remains challenging to precisely predict the functional H-L pairs and their interaction with antigens in silico. In previous studies, this issue was addressed with an additional phage display or a thorough screening to cover all possible combinations^{104, 105}. By understanding and refining the knowledge about residual preference in CDR3, as well as the findings that functional mAbs in antigen-affinity purified pAbs are of low variance in clonality, which underlie the rationale for selecting candidates based on a phylogenetic tree when generating a prior pool¹⁰⁴⁻¹⁰⁶, we have established the approach SPRP to help determine H-L pairs based on antibody sequences. With SPRP, the H-L pairs in a prior pool for testing could be reduced to as low as 13% of the entire possible combinations, and functional rAbs were successfully identified from all of the fusion clones. Compared with regular approaches, SPRP provided a quick, economic, and effective solution to the H-L pairing question. Although we could not reject the possibility that the optimal rAb was not included in a prior pool, because the antibody heavy or light chain genes in a fusion clone was commonly no more than 10 in our experiments, suggesting that it contains a very limited number of distinct antibody-expressing clones, it is unlikely that there is another high-activity antibody encoded by a cognate H-L chain pair outside the prior pool.

In Bio-Plex assays, the sensitivity, specificity, and dynamic range are highly influenced by the compatibility of the C-D antibodies. Among the factors that affect the compatibility, steric hindrance is a critical negative factor that is commonly found in antibody generation and use¹⁰⁷⁻¹¹³. In our study, results of the structure-based computational simulation were consistent with those of the antibody spatial blocking in vitro, which suggested that the compatibility of rabbit rAbs might be predicable in silico prior to antibody production and pairwise testing. In addition, our results showed that replacing murine mAbs and rabbit pAbs with rabbit rAbs could significantly improve both the sensitivity and the dynamic range.

Today, antibody-based applications and precision diagnostics call for a high standard of antibodies in terms of sensitivity and reliability¹¹⁴. Rabbit rAbs are excellent choice to meet the emerging needs of academia and industry. Our new method provides a convenient and cost-efficient alternative to current methods of generating rabbit rAbs in order to offer quality antibodies for improving Bio-Plex, as well as other antibody-based applications, from fundamental research to diagnostics.

### Detailed Description of Methodology Employed in Technical Section

The gene encoding a mouse IL-6R alpha protein truncation covering residues from number 20 to number 357 was amplified from a clone vector carrying its cDNA (Sinobiological Ltd. Co., Beijing, China) and was inserted into a modified pcDNA3.4 plasmid flanked by restriction sites SfiI and BamHI. This plasmid was termed pcDNA3.4-CD33-His-SIL-6Ra (20-357).

The genes encoding CH and CL of rabbit antibody were cloned from rabbit splencoytes by conventional reverse transcription polymerase chain reaction (RT-PCR). Then, they were inserted into modified pcDNA3.4 vector at restriction sites NheI and EcoRI to generate two expression vectors, which were termed pcDNA3.4-rab-IgH and pcDNA3.4-rab-IgL. These two expression vector were built to harbor VH and VL genes for expressing full-length rabbit rAbs. When constructing expression plasmids, the chosen VH gene was subcloned into pcDNA3.4-rab-IgH via restriction sites NheI and Eco91I, and the chosen VL gene was subcloned into pcDNA3.4-rab-IgL via restriction sites NheI and OliI.

### Immunogen Preparation

The recombinant mouse sIL-6Ra was expressed by transient transfection of Expi293 cells with the pcDNA3.4-CD33-His-sIL-6Ra (20-357) plasmid. The protein was purified from the cell culture supernatant by NTA affinity chromatography (Bio-Rad, Cat #7800801). Its purity was determined in reducing SDS-PAGE.

### Rabbit Immunization

Two New Zealand white rabbits weighing 2.5-3.0 kg were immunized subcutaneously. For a primary immunization, 500 μg recombinant sIL-6Rα protein was administered with Freund's complete adjuvant to each rabbit. For a booster immunization, three doses of 250 μg protein were administered with Freund's incomplete adjuvant at 2 weeks, 3 weeks and 4 weeks after the primary immunization. Blood and spleen were collected in 1 week after the final immunization. Suspension of the splenocytes was cryopreserved with 90% FBS and 10% DMSO by gradually cooling procedure and stored in liquid nitrogen. The rabbit immunization and splenocytes preparation described above was carried out by a commercial contractor (HuaBio Ltd. Co, Hangzhou, China)

### Cell Hybridization

The cell hybridization was achieved by electrofusion to generate heterohybridomas following a modified procedure. Before electrofusion, 2×10⁷ Sp2/0 mouse myeloma cells of 90% cell viability were collected and washed twice using RPMI-1640 medium. Cell suspension of rabbit splenocytes was prepared after removing the residual tissue and washing the cells twice using RPMI-1640 medium. The viability of Sp2/0 and rabbit splenocytes were determined by a cell counter (TC10, Bio-Rad) following the manufacturer's instruction. 6×10⁷ rabbit splenocytes were mixed with 2×10⁷ Sp2/0 cells and were spun down by centrifugation at 200 g for 5 min. The cell pellet was washed with 5 mL electrofusion buffer (0.28M sorbitol, 0.5 mM magnesium acetate, 0.1 mM calcium acetate, 1 mg/ml BSA) and suspended with fresh electrofusion buffer to reach 2 mL cell suspension for the electrofusion. Electric pulses were delivered with an electro cell manipulator (ECM2001, BTX). Parameters were determined in previous experiments: 40V for 25 s to line up the cells and 2000V for 30 us to fuse the cells. After delivery of pulses, cells were left undisturbed for 3 min. Then the cells was moved to 38 mL 2×HAT selection medium (2×HAT, 15% FBS, RPMI-1640). The cell suspension was dispensed into 96-w microplate at 100 μL per well, in which 100 μL suspension of mouse peritoneal exudate cells was added into as feeder cells. The cells were cultured at 37° C., 5% CO2. Half volume of the medium was changed with fresh medium at day 3 and all the medium was refreshed at day 6 with HAT selection medium. Approximately one week later, the cell clones formed and they were ready for ELISA screening.

### Screening Assay

When preparing ELISA assay plates, the recombinant mouse sIL-6Ra (20-357) was diluted to a final concentration of 1 μg/mL in a coating buffer (50 mM Sodium Carbonate, pH 9.6) to prepare a coating solution. The coating solution was dispensed into 96-w clean microplates (Costar #3590) at 100 μL/well for overnight incubation at 4° C. Then, the microplates were washed three times with PBST (0.05% Tween-20 in PBS) followed by a blocking solution (10% NCS/PBS) at 150 μL/well. The microplates were incubated at 37° C. for 2 hours. After blocking, the microplates were washed three times with PBST before use.

In an ELISA assay, supernatants of heterohybridoma culture were added into the designated wells at 100 μL/well. After incubation at 37° C. for 1 hours, the microplates were washed three times with PBST. A secondary goat-anti-rabbit antibody (KPI, Cat #Apr. 15, 2006 was diluted in PBST and the dilution was dispensed into each well at 100 μL for half hour incubation at 37° C. Then the microplates were washed three times with PBST again. After the wash, TMB solution (Sigma, Cat #860336) was dispensed into each well at 100 μL. After 5 minutes incubation at room temperature, the chromogenic reaction was terminated by adding 2M H₂SO₄ at 50 μL/well, and the optical density (OD) value of each well in the microplates were acquired by a microplate absorbance reader (Bio-Rad, Cat #1681135).

### Generating Prior Pools by Employing the Approach of Sequence Priority by Residual Preference (SPRP)

To clone the genes encoding VH and VL of rabbit antibodies from heterohybridoma cells, the total RNAs were isolated from the cells using RNeasy Micro Kit (QIAGEN, Cat #74004). Their cDNAs were reverse-transcriptionally synthesized using SMARTer® RACE 5′/3′ Kit (Clontech, Cat #634858). The VH and VL genes were amplified from the cDNAs by polymerase chain reaction (PCR) and subcloned into a modified pUC19 vector. The VH and VL gene sequences in these molecular clones were determined by Sanger sequencing method (Bioligo Ltd. Co., Shanghai, China).

Multiple sequence alignments of the amino acid sequences of VH regions of the amino acid sequences of VL regions were carried out using MUSCLE (https www ebi.ac.uk/Tools/msa/muscle/) and phylogenetic trees were established subsequently. Then, based on the alignments and phylogenetic trees, 1 sequence from each clade was selected by the following criteria:
a) CDR3 possesses moderate length (numbering IMGT) of 8-20 residues.
b) Exclude V regions containing a long tandem repeat (N>=4) in CDR3
c) Preference of residue Y, especially in the middle of CDR3 and spaced by G and S
d) These residues are not favored in CDR3: A, V, L, P, C, Q, K. Especially P, C, K and Q. Exclude V regions if its CDR3 contains more than 3 of P, C, K or Q.
e) for comparable V regions, pick one randomly. This step may also be optional
f) Determine the array to make the combination of H-L In some embodiments, <=12 in prior pool.

Empirically, it's most common to find one functional antibody from a positive fusion clone. Therefore, antibody amino acid sequences were aligned and the phylograms were used to visualize the diversity, which helped to compartmentalize the comparisons.

To construct expression plasmids, the selected V_{H} genes were subcloned into vector pcDNA3.4-rab-IgH via restriction sites NheI and Eco91I, and the selected V_{L} genes were subcloned into vector pcDNA3.4-rab-IgL via restriction sites NheI and OliI.

### Expression of Recombinant Antibodies

The plasmids were prepared from E. coli bacteria culture using a QIAprep Spin Miniprep Kit (Qiagen, Cat #27104). Plasmids carrying genes encoding antibody heavy chain and light chain were introduced into Expi293 cells using an ExpiFectamine™ 293 Transfection Kit (Life, Cat #A14524). The supernatants were collected after 5 days culture and filtrated through 0.45 μm filter. Then they were transferred to a ProteinA column (Bio-rad, Cat #732-4200) for affinity chromatography purification following the manufacturer's instruction. The protein concentration of purified recombinant antibodies was determined using a Nanodrop (Thermo, Cat #ND-2000).

### Validation of Recombinant Antibodies

The detection and capture reagents were prepared by covalently conjugating the purified antibodies to Biotin and resin, respectively. They were paired and tested in Bio-Plex assays to determine optimal C-D antibody pairs. The rest buffers and reagents used in Bio-Plex assays were from a commercial kit (Bio-Rad, Cat #171304030M). The coupled beads were prepared following the manufacturer's manual.

In each assay, 50 μL capture reagent was added into a Bio-Plex microplate, and the microplate was washed twice using the wash buffer. Then, 50 μL antigen at indicated concentration was added into the designated wells, and the microplate was incubated at room temperature for 1 hour. After the incubation, the antigen was removed and the microplate was washed twice. 50 μL 2 μg/mL biotin-conjugated monoclonal antibody were added into the wells and incubated at room temperature for 1 hour. After the incubation, the microplate was washed twice. Following that, 50 μL 1× streptavidin-PE solution was added into the wells and incubated for 10 min. After removing the streptavidin-PE solution, the microplate was washed three time and subjected to a Bio-Plex reader to acquire the reads (Bio-Rad, Bio-Plex-200)

### Bioinformatic Analysis

The DNA sequence of rabbit antibody genes were analyzed using an online tool to identify the productive ones and determine the residues in third complementarity determining region (CDR3) (www imgt.org)^{1, 2}. Their amino acid sequences were aligned online for studying the diversity (www ebi.ac.uk/Tools/msa/muscle/)³.

In an in silico simulation, the mouse sIL-6Ra structure model was computationally predicted based on a X-ray crystal structure of the extracellular domain of human interleukin-6 receptor alpha chain (SMTL ID: In26.1)⁴ (swissmodel.expasy.org/interactive)⁵⁻⁹. The structure models of variable fragments (Fv) of rabbit rAbs (rAb #22, rAb #52 and rAb #62) were predicted and built based on their amino acid sequences (opig.stats.ox.ac.uk/webapps/newsabdab/sabpred/abodybuilder/)¹⁰. An docking tool was used to analyze the antigen-antibody interaction based on the prediction models of mouse sIL-6Ra and rabbit rAbs (http://frodock.chaconlab.org/)¹¹. The top 1 solution of each simulation was taken to present the antigen-antibody docking (See FIG. 5A).

### Antigen-Antibody Affinity Measurement

The antigen-antibody affinity of four antibodies (rAb #22, rAb #52, rAb #62, a rat mAb RB01) were determined by an analysis service employing a surface plasmon resonance technology (Beijing Xtalbio Ltd. Co). It started with a directly immobilization of recombinant mouse sIL-6Ra (20-357) on a CM5 S sensor chip using Biacore® S200 machine. Approximately 27.8 resonance units (RU) of the sIL-6Ra (20-357) were immobilized at 25° C. with an amine coupling kit following the standard amine coupling procedure. The remaining active sites on the sensor chip were blocked with 1 M ethanolamine-HCl solution, pH 8.5 at a flow rate of 10 μL/min for 7 min, to finally reach 27.8 RU. The flow cells were washed with the injection of HBS-EP running buffer at 30 μL/min. For each antibody, a 2-fold serial dilution (0, 0.39 nM-200 nM) was injected at 15° C. to a channel with a flow rate of 30 μL/min for 120s and dissociation was followed for 720s. The curves were locally fitted with Biacore®8K analysis software (BIAevaluation) using a 1:1 Langmuir binding model.

Using the same technology, a competitive assay was set up to analyze the competitive bindings of rAb #22, rAb #52 and rAb #62. Approximately 49.7 resonance units (RU) of recombinant mouse sIL-6Ra (20-357) were immobilized at 25° C. on a CM5 chip with an amine coupling kit following the standard amine coupling procedure. The sIL-6Ra (20-357) was diluted to 2 μg/mL in 10 mM immobilization buffer (10 mM Na Acetate pH5.0) and injected only to flow cell of channel 1 and 2 at a flow rate of 5 μL/min. The remaining active sites on the sensor chip were blocked with 1 M ethanolamine-HCl solution, pH 8.5 at a flow rate of 10 μL/min for 7 min, to finally reach 49.7 RU. The entire test for each antibody sample split into 3 cycles: in the first cycle, HBS-EP buffer & 200 nM of 1st antibody were injected in order at 25° C. to channel 1 & 2 with a flow rate of 30 μL/min for 90s and stabilized for 60s; in the second cycle, 200 nM of 2nd & 1st antibodies were injected in order at 25° C. to channel 1 & 2 with a flow rate of 30 μL/min for 90s and stabilized for 60s; in the third cycle, 200 nM of 2nd antibody & HBS-EP buffer were injected in order at 25° C. to channel 1 & 2 with a flow rate of 30 μL/min for 90s and stabilized for 60s. The three curves were merged together using Biacore®T200 analysis software (Biacore®T200 BIAevaluation) (See FIG. 5D-I).

### REFERENCES CITED BY NUMBER IN METHODOLOGY SECTION

1. Brochet X, Lefranc M-P, Giudicelli V. IMGT/V-QUEST: the highly customized and integrated system for IG and TR standardized VJ and VDJ sequence analysis. Nucleic Acids Res. 2008; 36(suppl_2):W503-W508.
2. Giudicelli V, Brochet X, Lefranc M-P. IMGT/V-QUEST: IMGT standardized analysis of the immunoglobulin (IG) and T cell receptor (TR) nucleotide sequences. Cold Spring Harb Protoc. 2011; 2011(6):pdb-prot5633.
3. Madeira F, Park Y M, Lee J, et al. The EMBL-EBI search and sequence analysis tools APIs in 2019. Nucleic Acids Res. 2019; 47(W1):W636-W641.
4. Varghese J N, Moritz R L, Lou M-Z, et al. Structure of the extracellular domains of the human interleukin-6 receptor α-chain. Proc Natl Acad Sci. 2002; 99(25):15959-15964.
5. Waterhouse A, Bertoni M, Bienert S, et al. SWISS-MODEL: homology modelling of protein structures and complexes. Nucleic Acids Res. 2018; 46(W1):W296-W303.
6. Bienert S, Waterhouse A, de Beer T A P, et al. The SWISS-MODEL Repository—new features and functionality. Nucleic Acids Res. 2017; 45(D1):D313-D319.
7. Guex N, Peitsch M C, Schwede T. Automated comparative protein structure modeling with SWISS-MODEL and Swiss-PdbViewer: A historical perspective. Electrophoresis. 2009; 30(S1):S162-S173.
8. Benkert P, Biasini M, Schwede T. Toward the estimation of the absolute quality of individual protein structure models. Bioinformatics. 2011; 27(3):343-350.
9. Bertoni M, Kiefer F, Biasini M, Bordoli L, Schwede T. Modeling protein quaternary structure of homo- and hetero-oligomers beyond binary interactions by homology. Sci Rep. 2017; 7(1):1-15.
10. Leem J, Dunbar J, Georges G, Shi J, Deane C M. ABody Builder: Automated antibody structure prediction with data-driven accuracy estimation. In: MAbs. Vol 8. Taylor & Francis; 2016:1259-1268.
11. Ramírez-Aportela E, López-Blanco J R, Chacón P. FRODOCK 2.0: fast protein-protein docking server. Bioinformatics. 2016; 32(15):2386-2388.

### REFERENCES OTHER THAN THOSE CITED IN THE METHODS SECTION CITED BY NUMBER IN THE APPLICATION

1. Köhler, G. & Milstein, C. Continuous cultures of fused cells secreting antibody of predefined specificity. Nature 256, 495-497 (1975).
2 Nelson, P. N. et al. Demystified . . . : monoclonal antibodies. Mol. Pathol. 53, 111 (2000).
3. Liu, J. K. H. The history of monoclonal antibody development-progress, remaining challenges and future innovations. Ann. Med. Surg. 3, 113-116 (2014).
4. Hurrell, J. G. R. Monoclonal hybridoma antibodies: techniques and applications. (CRC press Boca Raton, FL, 1982).
5. Rocha, R. et al. Rabbit monoclonal antibodies show higher sensitivity than mouse monoclonals for estrogen and progesterone receptor evaluation in breast cancer by immunohistochemistry. Pathol. Pract. 204, 655-662 (2008).
6. Terasawa, K. et al. Preferential recognition of isocitrate dehydrogenase by a rabbit monoclonal antibody (ab124797) against the C-terminal peptide of RANKL. J. Immunol. Methods 420, 1-10 (2015).
7 Weber, J., Peng, H. & Rader, C. From rabbit antibody repertoires to rabbit monoclonal antibodies. Exp. Mol. Med. 49, e305-e305 (2017).
8. Liang, Z., La Scola, B. & Raoult, D. Monoclonal antibodies to immunodominant epitope of Tropheryma whipplei. Clin. Diagn. Lab. Immunol. 9, 156-159 (2002).
9. Yu, M. et al. Determination and application of immunodominant regions of SARS coronavirus spike and nucleocapsid proteins recognized by sera from different animal species. J. Immunol. Methods 331, 1-12 (2008).
10. He, Y. et al. Identification of immunodominant sites on the spike protein of severe acute respiratory syndrome (SARS) coronavirus: implication for developing SARS diagnostics and vaccines. J. Immunol. 173, 4050-4057 (2004).
11. Liu, C.-C. et al. Identification and characterization of a cross-neutralization epitope of Enterovirus 71. Vaccine 29, 4362-4372 (2011).
12. Bystryn, J.-C., JACOBSEN, J. S., Liu, P. & Heaney-Kieras, J. Comparison of cell-surface human melanoma-associated antigens identified by rabbit and murine antibodies. Hybridoma 1, 465-472 (1982).
13. Rossi, S. et al. Rabbit monoclonal antibodies: A comparative study between a novel category of immunoreagents and the corresponding mouse monoclonal antibodies. Am. J. Clin. Pathol. (2005) doi:10.1309/N8HN08GDPVEMU08.
14. Feng, L., Wang, X. & Jin, H. Rabbit monoclonal antibody: potential application in cancer therapy. Am. J. Transl. Res. 3, 269 (2011).
15. Rader, C. et al. The rabbit antibody repertoire as a novel source for the generation of therapeutic human antibodies. J. Biol. Chem. 275, 13668-13676 (2000).
16. Borras, L. et al. Generic approach for the generation of stable humanized single-chain Fv fragments from rabbit monoclonal antibodies. J. Biol. Chem. 285, 9054-9066 (2010).
17. Yu, Y. et al. A humanized anti-VEGF rabbit monoclonal antibody inhibits angiogenesis and blocks tumor growth in xenograft models. PLOS One 5, (2010).
18. Cheah, A. L. et al. STAT6 rabbit monoclonal antibody is a robust diagnostic tool for the distinction of solitary fibrous tumour from its mimics. Pathology 46, 389-395 (2014).
19. Cheuk, W., Wong, K. O. Y., Wong, C. S. C. & Chan, J. K. C. Consistent immunostaining for cyclin D1 can be achieved on a routine basis using a newly available rabbit monoclonal antibody. Am. J. Surg. Pathol. 28, 801-807 (2004).
20. Zhu, W., Li, A. & Chen, T. Rabbit monoclonal antibodies. in Handbook of Practical Immunohistochemistry 77-84 (Springer, 2015).
21. Lin, F. & Prichard, J. Handbook of practical immunohistochemistry: frequently asked questions. (Springer, 2015).
22. Markham, A. Brolucizumab: first approval. Drugs 79, 1997-2000 (2019).
23. Song, S.-W. et al. Inhibition of tumor growth in a mouse xenograft model by the humanized anti-HGF monoclonal antibody YYB-101 produced in a large-scale CHO cell culture. J Microbiol Biotechnol 23, 1327-1338 (2013).
24. Yang, J. et al. Therapeutic potential and challenges of targeting receptor tyrosine kinase ROR1 with monoclonal antibodies in B-cell malignancies. PLOS One 6, e21018 (2011).
25. Berger, C. et al. Safety of targeting ROR1 in primates with chimeric antigen receptor-modified T cells. Cancer Immunol. Res. 3, 206-216 (2015).
26. Raybould, T. J. & Takahashi, M. Production of stable rabbit-mouse hybridomas that secrete rabbit mAb of defined specificity. Science (80-.). 240, 1788-1790 (1988).
27. Mei-Chang, K., Soon, J. A., Max, E. E. & Kindt, T. J. Rabbit-mouse hybridomas secreting intact rabbit immunoglobulin. Mol. Immunol. (1985) doi: 10.1016/0161-5890 (85) 90119-1.
28. Campbell, A. M. Monoclonal antibody technology: the production and characterization of rodent and human hybridomas. (Elsevier, 2000).
29. Erikson, J., Martinis, J. & Croce, C. M. Assignment of the genes for human λ immunoglobulin chains to chromosome 22. Nature 294, 173-175 (1981).
30. Tucker, E. M., Dain, A. R., Clarke, S. W. & Donker, R. A. Specific bovine monoclonal antibody produced by a re-fused mouse/calf hybridoma. Hybridoma 3, 171-176 (1984).
31. Flynn, J. N., Harkiss, G. D. & Hopkins, J. Generation of a sheepx mouse heterohybridoma cell line (1C6. 3a6T. 1D7) and evaluation of its use in the production of ovine monoclonal antibodies. J. Immunol. Methods 121, 237-246 (1989).
32. JAMES, D. E., KREUTZ, F. T., BIGGS, D. F. & SURESH, M. R. Production of murine/guinea pig and rat/guinea pig heterohybridomas. Hybridoma 15, 387-389 (1996).
33. Yarmush, M. L., Gates, F. T., Weisfogel, D. R. & Kindt, T. J. Identification and characterization of rabbit-mouse hybridomas secreting rabbit immunoglobulin chains. Proc. Natl. Acad. Sci. 77, 2899-2903 (1980).
34. Dreher, K. L., Sogn, J. A., Gates, F. T., Kuo, M. C. & Kindt, T. J. Allotype-defined mRNA for rabbit immunoglobulin H and L chains isolated from rabbit-mouse hybridomas. J. Immunol. 130, 442-448 (1983).
35. Verbanac, K. M., Gross, U. M., Rebellato, L. M. & Thomas, J. M. Production of stable rabbit-mouse heterohybridomas: Characterization of a rabbit monoclonal antibody recognizing a 180 kDa human lymphocyte membrane antigen. Hybridoma 12, 285-295 (1993).
36. Raman, C., Spieker-Polet, H., Yam, P.-C. & Knight, K. L. Preferential VH gene usage in rabbit Ig-secreting heterohybridomas. J. Immunol. 152, 3935-3945 (1994).
37. Groves, D. J. & Morris, B. A. Veterinary sources of nonrodent monoclonal antibodies: interspecific and intraspecific hybridomas. Hybridoma 19, 201-214 (2000).
38. Spieker-Polet, H., Sethupathi, P., Yam, P.-C. & Knight, K. L. Rabbit monoclonal antibodies: generating a fusion partner to produce rabbit-rabbit hybridomas. Proc. Natl. Acad. Sci. 92, 9348-9352 (1995).
39. McCormack, R. T., Liu, R., Manetta, J. V & Sportsman, J. R. Stable rabbit-mouse fusion partner. (1995).
40. Knight, K. L. Immortalized rabbit hybridoma fusion partner. (1997).
41. Pytela, R., Zhu, W., Ke, Y., Qian, Q. & Au, H. C. Fusion partner for production of monoclonal rabbit antibodies. (2008).
42. Pytela, R., Zhu, W., Ke, Y., Qian, Q. & Au, H. C. Fusion partner for production of monoclonal rabbit antibodies. (2016).
43. Liguori, M. J., Hoff-Velk, J. A. & Ostrow, D. H. Recombinant human interleukin-6 enhances the immunoglobulin secretion of a rabbit-rabbit hybridoma. Hybridoma 20, 189-198 (2001).
44. Frame, K. K. & Hu, W. The loss of antibody productivity in continuous culture of hybridoma cells. Biotechnol. Bioeng. 35, 469-476 (1990).
45. OZTURK, S. S. & ØPALSSON, B. Loss of antibody productivity during long-term cultivation of a hybridoma cell line in low serum and serum-free media. Hybridoma 9, 167-175 (1990).
46. Merritt, S. E. & Palsson, B. O. Loss of antibody productivity is highly reproducible in multiple hybridoma subclones. Biotechnol. Bioeng. 42, 247-250 (1993).
47. Bradbury, A. R. M. & Plückthun, A. Getting to reproducible antibodies: the rationale for sequenced recombinant characterized reagents. Protein Eng. Des. Sel. 28, 303-305 (2015).
48. Bradbury, A. & Plückthun, A. Reproducibility: Standardize antibodies used in research. Nature 518, 27-29 (2015).
49. Bradbury, A. R. M. et al. When monoclonal antibodies are not monospecific: Hybridomas frequently express additional functional variable regions. MAbs 10, 539-546 (2018).
50. Baker, M. Blame it on the antibodies. Nature 521, 274 (2015).
51. Hell, S. W. et al. The 2015 super-resolution microscopy roadmap. J. Phys. D. Appl. Phys. 48, 443001 (2015).
52. Weller, M. G. Quality issues of research antibodies. Anal. Chem. Insights 11, ACI-S31614 (2016).
53. Bordeaux, J. et al. Antibody validation. Biotechniques 48, 197-209 (2010).
54. Smith, G. P. & Petrenko, V. A. Phage display. Chem. Rev. 97, 391-410 (1997).
55. Clackson, T., Hoogenboom, H. R., Griffiths, A. D. & Winter, G. Making antibody fragments using phage display libraries. Nature 352, 624-628 (1991).
56. Barbas, C. F., Kang, A. S., Lerner, R. A. & Benkovic, S. J. Assembly of combinatorial antibody libraries on phage surfaces: the gene III site. Proc. Natl. Acad. Sci. 88, 7978-7982 (1991).
57. Winter, G., Griffiths, A. D., Hawkins, R. E. & Hoogenboom, H. R. Making antibodies by phage display technology. Annu. Rev. Immunol. 12, 433-455 (1994).
58. Rader, C. & Barbas III, C. F. Phage display of combinatorial antibody libraries. Curr. Opin. Biotechnol. 8, 503-508 (1997).
59. Rader, C. Antibody libraries in drug and target discovery. Drug Discov. Today 6, 36-43 (2001).
60. Ridder, R., Schmitz, R., Legay, F. & Gram, H. Generation of rabbit monoclonal antibody fragments from a combinatorial phage display library and their production in the yeast Pichia pastoris. Bio/technology 13, 255-260 (1995).
61. Chi, X. S., Landt, Y., Crimmins, D. L., Dieckgraefe, B. K. & Ladenson, J. H. Isolation and characterization of rabbit single chain antibodies to human Reg Iα protein. J. Immunol. Methods 266, 197-207 (2002).
62. Moon, S. A. et al. Antibodies against non-immunizing antigens derived from a large immune scFv library. Mol. Cells 31, 509-513 (2011).
63. Lang, I. M., Barbas III, C. F. & Schleef, R. R. Recombinant rabbit Fab with binding activity to type-1 plasminogen activator inhibitor derived from a phage-display library against human α-granules. Gene 172, 295-298 (1996).
64. Foti, M. et al. Rabbit monoclonal Fab derived from a phage display library. J. Immunol. Methods 213, 201-212 (1998).
65. Li, Y., Cockburn, W., Kilpatrick, J. B. & Whitelam, G. C. High affinity ScFvs from a single rabbit immunized with multiple haptens. Biochem. Biophys. Res. Commun. 268, 398-404 (2000).
66. Hudson, P. J. & Kortt, A. A. High avidity scFv multimers; diabodies and triabodies. J. Immunol. Methods 231, 177-189 (1999).
67. Rader, C. Generation and selection of rabbit antibody libraries by phage display. in Therapeutic Antibodies 101-128 (Springer, 2009).
68. Popkov, M., Rader, C. & Barbas III, C. F. Isolation of human prostate cancer cell reactive antibodies using phage display technology. J. Immunol. Methods 291, 137-151 (2004).
69. Nguyen, T. T. H., Lee, J. S. & Shim, H. Construction of rabbit immune antibody libraries. in Phage Display 133-146 (Springer, 2018).
70. He, B. et al. Compositional bias in naïve and chemically-modified phage-displayed libraries uncovered by paired-end deep sequencing. Sci. Rep. 8, 1-14 (2018).
71. Matochko, W. L., Cory Li, S., Tang, S. K. Y. & Derda, R. Prospective identification of parasitic sequences in phage display screens. Nucleic Acids Res. 42, 1784-1798 (2014).
72. Rodi, D. J., Soares, A. S. & Makowski, L. Quantitative assessment of peptide sequence diversity in M13 combinatorial peptide phage display libraries. J. Mol. Biol. 322, 1039-1052 (2002).
73. Seeber, S. et al. A robust high throughput platform to generate functional recombinant monoclonal antibodies using rabbit B cells from peripheral blood. PLOS One 9, (2014).
74. Rashidian, J. et al. Generation and Performance of R132H Mutant IDH1 Rabbit Monoclonal Antibody. Antibodies 6, 22 (2017).
75. Kurosawa, N., Yoshioka, M., Fujimoto, R., Yamagishi, F. & Isobe, M. Rapid production of antigen-specific monoclonal antibodies from a variety of animals. BMC Biol. 10, 80 (2012).
76. Jin, A. et al. Rapid isolation of antigen-specific antibody-secreting cells using a chip-based immunospot array. Nat. Protoc. 6, 668-676 (2011).
77. Jin, A. et al. A rapid and efficient single-cell manipulation method for screening antigen-specific antibody-secreting cells from human peripheral blood. Nat. Med. 15, 1088-1092 (2009).
78. Love, J. C., Ronan, J. L., Grotenbreg, G. M., van der Veen, A. G. & Ploegh, H. L. A microengraving method for rapid selection of single cells producing antigen-specific antibodies. Nat. Biotechnol. 24, 703-707 (2006).
79. Ogunniyi, A. O., Story, C. M., Papa, E., Guillen, E. & Love, J. C. Screening individual hybridomas by microengraving to discover monoclonal antibodies. Nat. Protoc. 4, 767 (2009).
80. Starkie, D. O., Compson, J. E., Rapecki, S. & Lightwood, D. J. Generation of recombinant monoclonal antibodies from immunised mice and rabbits via flow cytometry and sorting of antigen-specific IgG+ memory B cells. PLOS One 11, e0152282 (2016).
81. Murakami, M. et al. IL-6-induced homodimerization of gp130 and associated activation of a tyrosine kinase. Science (80-.). 260, 1808-1810 (1993).
82. Rose-John, S. IL-6 trans-signaling via the soluble IL-6 receptor: importance for the pro-inflammatory activities of IL-6. Int. J. Biol. Sci. 8, 1237 (2012).
83. Riethmueller, S. et al. Cleavage site localization differentially controls interleukin-6 receptor proteolysis by ADAM10 and ADAM17. Sci. Rep. 6, 25550 (2016).
84. Schumacher, N. et al. Shedding of endogenous interleukin-6 receptor (IL-6R) is governed by a disintegrin and metalloproteinase (ADAM) proteases while a full-length IL-6R isoform localizes to circulating microvesicles. J. Biol. Chem. 290, 26059-26071 (2015).
85. Garbers, C. et al. Species specificity of ADAM10 and ADAM17 proteins in interleukin-6 (IL-6) trans-signaling and novel role of ADAM10 in inducible IL-6 receptor shedding. J. Biol. Chem. 286, 14804-14811 (2011).
86. Haddick, P. C. G. et al. A Common variant of IL-6R is associated with elevated IL-6 pathway activity in Alzheimer's Disease brains. J. Alzheimer's Dis. 56, 1037-1054 (2017).
87. Garbers, C. & Rose-John, S. Dissecting interleukin-6 classic- and trans-signaling in inflammation and cancer. in Inflammation and Cancer 127-140 (Springer, 2018).
88. Atreya, R. et al. Blockade of interleukin 6 trans signaling suppresses T-cell resistance against apoptosis in chronic intestinal inflammation: evidence in crohn disease and experimental colitis in vivo. Nat. Med. 6, 583-588 (2000).
89. Matsumoto, S. et al. Essential roles of IL-6 trans-signaling in colonic epithelial cells, induced by the IL-6/soluble-IL-6 receptor derived from lamina propria macrophages, on the development of colitis-associated premalignant cancer in a murine model. J. Immunol. 184, 1543-1551 (2010).
90. Jones, S. A., Horiuchi, S., Topley, N., Yamamoto, N. & Fuller, G. M. The soluble interleukin 6 receptor: mechanisms of production and implications in disease. FASEB J. 15, 43-58 (2001).
91. Rossi, J.-F., Lu, Z.-Y., Jourdan, M. & Klein, B. Interleukin-6 as a therapeutic target. Clin. Cancer Res. 21, 1248-1257 (2015).
92. De Benedetti, F. et al. Serum soluble interleukin 6 (IL-6) receptor and IL-6/soluble IL-6 receptor complex in systemic juvenile rheumatoid arthritis. J. Clin. Invest. 93, 2114-2119 (1994).
93. Suzuki, H. et al. Serum soluble interleukin-6 receptor in MRL/lpr mice is elevated with age and mediates the interleukin-6 signal. Eur. J. Immunol. 23, 1078-1082 (1993).
94. Peters, M., zum Büschenfelde, K.-H. M. & Rose-John, S. The function of the soluble IL-6 receptor in vivo. Immunol. Lett. 54, 177-184 (1996).
95. Wunderlich, C. M. et al. Cutting edge: Inhibition of IL-6 trans-signaling protects from malaria-induced lethality in mice. J. Immunol. 188, 4141-4144 (2012).
96. Mancini, S. J. et al. Activation of AMP-activated protein kinase rapidly suppresses multiple pro-inflammatory pathways in adipocytes including IL-1 receptor-associated kinase-4 phosphorylation. Mol. Cell. Endocrinol. 440, 44-56 (2017).
97. Robin, G. et al. Restricted diversity of antigen binding residues of antibodies revealed by computational alanine scanning of 227 antibody-antigen complexes. J. Mol. Biol. 426, 3729-3743 (2014).
98. Fellouse, F. A. et al. Molecular recognition by a binary code. J. Mol. Biol. 348, 1153-1162 (2005).
99. Fellouse, F. A. et al. High-throughput generation of synthetic antibodies from highly functional minimalist phage-displayed libraries. J. Mol. Biol. 373, 924-940 (2007).
100. Birtalan, S. et al. The intrinsic contributions of tyrosine, serine, glycine and arginine to the affinity and specificity of antibodies. J. Mol. Biol. 377, 1518-1528 (2008).
101. Nguyen, M. N., Pradhan, M. R., Verma, C. & Zhong, P. The interfacial character of antibody paratopes: analysis of antibody-antigen structures. Bioinformatics 33, 2971-2976 (2017).
102. Padlan, E. A. Anatomy of the antibody molecule. Mol. Immunol. 31, 169-217 (1994).
103. AlQuraishi, M. AlphaFold at CASP13. Bioinformatics 35, 4862-4865 (2019).
104. Cheung, W. C. et al. A proteomics approach for the identification and cloning of monoclonal antibodies from serum. Nat. Biotechnol. 30, 447-452 (2012).
105. Wine, Y. et al. Molecular deconvolution of the monoclonal antibodies that comprise the polyclonal serum response. Proc. Natl. Acad. Sci. 110, 2993-2998 (2013).
106. Boutz, D. R. et al. Proteomic identification of monoclonal antibodies from serum. Anal. Chem. 86, 4758-4766 (2014).
107. Stone, M. R. & Nowinski, R. C. Topological mapping of murine leukemia virus proteins by competition-binding assays with monoclonal antibodies. Virology 100, 370-381 (1980).
108. Massey, R. J. & Schochetman, G. Topographical analysis of viral epitopes using monoclonal antibodies: mechanism of virus neutralization. Virology 115, 20-32 (1981).
109. Koch, N., Hammerling, G. J., Tada, N., Kimura, S. & Hammerling, U. Cross-blocking studies with monoclonal antibodies against I-A molecules of haplotypes b, d and k. Eur. J. Immunol. 12, 909-914 (1982).
110. Labrijn, A. F. et al. Access of antibody molecules to the conserved coreceptor binding site on glycoprotein gp120 is sterically restricted on primary human immunodeficiency virus type 1. J. Virol. 77, 10557-10565 (2003).
111. Vilim, V. et al. Monoclonal antibodies to human cartilage oligomeric matrix protein: epitope mapping and characterization of sandwich ELISA. Clin. Chim. acta 328, 59-69 (2003).
112. Ndifon, W., Wingreen, N. S. & Levin, S. A. Differential neutralization efficiency of hemagglutinin epitopes, antibody interference, and the design of influenza vaccines. Proc. Natl. Acad. Sci. 106, 8701-8706 (2009).
113. Zhu, L. et al. Development of a double-antibody sandwich ELISA for rapid detection of Bacillus Cereus in food. Sci. Rep. 6, 1-10 (2016).
114. Borrebaeck, C. A. K. Precision diagnostics: moving towards protein biomarker signatures of clinical utility in cancer. Nat. Rev. Cancer 17, 199 (2017).

Illustrative Sequences

SEQ ID NO: 1 Antibody 22 Heavy Chain Variable
Region Protein Sequence
METGLRWLLLVAVLKGVQCQSLEESGGGLVQPEGSLTLTCKASGFSFSG
DYDMCWVRQAPGKGLEWIGCTDSGFSGTTYYASWAKGRFTISKTSSTTV
TLQTTSLTAADTATYFCARDFDSSSGYYWDLWGPGTLVTVSS
SEQ ID NO: 2 Antibody 22 Light Chain Variable
Region Protein Sequence
MDTRAPTQLLGLLLLWLPGATFAIVMTQTPSSKSVPVGDTVTINCQASE
SVSSNNRLAWYQQKPGQPPKLLIYAASTLTSGVPSRFKGSGSGTQFTLT
ISGVQCDDAATYYCAGYKGLYSDGRAFGGGTEVVVK
SEQ ID NO: 3 Antibody 48 Heavy Chain Variable
Region Protein Sequence
METGLRWLLLVAVLKGVQCQEQLVESGGGLVQPEGSLTLTCKASGFSFI
SYHYYMCWVRQAPGKGLEWIACIDAISSGSTYYASWAKGRVTISKTSST
TVTLQMTSLTAADTATYFCARAPYYTYDYVGYALTLWGPGTLVTVSS
SEQ ID NO: 4 Antibody 48 Light Chain Variable
Region Protein Sequence
MDTRAPTQLLGLLLLWLPGARCADIVMTQTPASVEAAVGGTVTINCQAS
ENIYNFLTWYQQKPGQRPKLLIYRASTLASGVPSRFKGSGSGTDFTLTI
SDLECADAATYYCQRNAYSSWAFGGGTEVVVK
SEQ ID NO: 5 Antibody 52 Heavy Chain Variable
Region Protein Sequence
METGLRWLLLVAVLKGVQCQSLEESGGDLVKPGASLTLACTASGFSFSS
GYYMCWVRQAPGKGLEWIGCIYTGSDTTYYASWAKGRFTISETSSTAAT
LQMTSLTAADTATYFCARDLGSRGNLWGPGTLVTVSS
SEQ ID NO: 6 Antibody 52 Light Chain Variable
Region Protein Sequence
MDTRAPTQLLGLLLLWLPGARCADIVMTQTPSSVSAAVGDTVTINCQAS
ENIYSFLTWYQQKPGQRPKLLIYRASTLASGVPSRFKGSGSGTDFTLTI
SDLECADAATYYCQRNAYSSWAFGGGTEVVVK
SEQ ID NO: 7 Antibody 62 Heavy Chain Variable
Region Protein Sequence
METGLRWLLLVAVLKGVQCQEHLEESGGGLVQPEGSLTLTCTASGFSFS
SNYWICWVRQAPGKGLEWIACIYLFSVGNTFYASWAKDRFTISRSTSLN
TVTLQMTSLTAADTATYFCARAPYYLDGYARAYAFNLWGPGTLVTVSS
SEQ ID NO: 8 Antibody 62 Light Chain Variable
Region Protein Sequence
MDTRAPTQLLGLLLLWLPGARCAIDMTQTPASLSAAVGGTVAINCQASQ
SIGSDLAWYQQKPGQPPKLLIYFASKLASGVSSRFKGSGSGTQFTLTIS
DLECDDVATYYCAGGYNSADIFAFGGGTEVVVK
SEQ ID NO: 9 sIL-6Rα polypeptide sequence
UniProt Ref. P08887-2
MLAVGCALLAALLAAPGAALAPRRCPAQEVARGVLTSLPGDSVTLTCPG
VEPEDNATVHWVLRKPAAGSHPSRWAGMGRRLLLRSVQLHDSGNYSCYR
AGRPAGTVHLLVDVPPEEPQLSCFRKSPLSNVVCEWGPRSTPSLTTKAV
LLVRKFQNSPAEDFQEPCQYSQESQKFSCQLAVPEGDSSFYIVSMCVAS
SVGSKFSKTQTFQGCGILQPDPPANITVTAVARNPRWLSVTWQDPHSWN
SSFYRLRFELRYRAERSKTFTTWMVKDLQHHCVIHDAWSGLRHVVQLRA
QEEFGQGEWSEWSPEAMGTPWTESRSPPAENEVSTPMQALTIN

1. A method of identifying a rabbit monoclonal antibody, the method comprising:
(a) generating heterohybridomas comprising splenocytes obtained from a rabbit immunized with an antigen of interest fused to mouse myeloma cell;
(b) screening heterohybridomas generated in (a) to identify antibodies that bind to the antigen of interest;
(c) selecting heterohybridomas that bind to the antigen of interest to provide a subpopulation for further screening
(d) obtaining V_{H} and V_{L} CDNAs from RNA isolated from each heterohybridoma identified in (b)
(e) sequencing the V_{H} and V_{L} CDNA, and aligning V_{H} sequences to prepare a phylogenetic tree for V_{H} regions and aligning V_{L} regions to prepare a phylogenetic tree for V_{L} regions for each heteorhybridoma;
(f) selecting a V_{H} region that comprises a CDR3 that is 8-20 residues in length; and
(i) does not contain greater than 3 consecutive residues of the same amino acid;
(ii) comprises a at least one aromatic residue;
(iii) does not comprise three of one of the following amino acids, or three of the following amino acids in combination: P, C, K or Q;
(g) selecting a V_{L} region that comprises a CDR3 that is 8-20 residues in length; and
(i) does not contain more than 3 consecutive residues of the same amino acid;
(ii) comprises an aromatic amino acid;
(iii) does not comprise three of one of the following amino acids, or three of the following amino acids in combination: P, C, K or Q;
(h) independently expressing a V_{H} region selected in (f) with each V_{L} region selected in (g) to provide V_{H} and V_{L} region pairs; and independently expressing each V_{L} region selected in (g) with each V_{H} region selected (f) to provide V_{H} and V_{L} pairs;
(i) analyzing the V_{H} and V_{L} region pairs to identify a pair that binds to the antigen of interest with the highest affinity relative to the other pairs.

2. The method of claim 1, wherein the HCDR3 and/or the LCDR3 comprises two or more aromatic residues, wherein two aromatic residues are separated by at least one G or S residue.

3. The method of claim 1, wherein (g) is performed before (f).

4. An anti-IL-6 receptor-alpha (IL-6Rα) antibody that binds to a soluble IL-6Rα (sIL-6Rα) polypeptide, said anti-IL-6Rα antibody comprising:
(a)
HCDR1:
(SEQ ID NO: 10)
GFSFSGDYD
HCDR2:
(SEQ ID NO: 11)
TDSGFSGTT
HCDR3:
(SEQ ID NO: 12)
ARDFDSSGSYYWDL
LCDR1:
(SEQ ID NO: 13)
ESVSSNNR
LCDR2:
AAS
LCDR3:
(SEQ ID NO: 14)
AGYKGLYSDGRA;
(b)
HCDR1:
(SEQ ID NO: 15)
GFSFISYHYY
HCDR2:
(SEQ ID NO: 16)
IDAISSGST
HCDR3:
(SEQ ID NO: 17)
ARAPYYTYDGVYYALTL
LCDR1:
(SEQ ID NO: 18)
ENIYNF
LCDR2:
RAS
LCDR3:
(SEQ ID NO: 19)
QRNAYSSWA;
(c)
HCDR1:
(SEQ ID NO: 20)
GFSFSSGYY
HCDR2:
(SEQ ID NO: 21)
IYTGSDTT
HCDR3:
(SEQ ID NO: 22)
ARDLGSRGNL
LCDR1:
(SEQ ID NO: 23)
ENIYSF
LCDR2:
RAS
LCDR3:
(SEQ ID NO: 19)
QRNAYSSWA;
or
(d)
HCDR1:
(SEQ ID NO: 24)
GFSFSSNYW
HCDR2:
(SEQ ID NO: 25)
IYLFSVGNT
HCDR3:
(SEQ ID NO: 26)
ARAPYYLDGARAYYAFNL
LCDR1:
(SEQ ID NO: 27)
QSIGSD
LCDR2:
FAS
LCDR3:
(SEQ ID NO: 28)
AGGYNSADIFA.

5. The antibody of claim 4; wherein:
the V_{H} region has at least 95% identity to the amino acid sequence set forth in SEQ ID NO:1 and the V_{L} region has at least 95% identity to the amino acid sequence set forth in SEQ ID NO:2;
the V_{H} region has at least 95% identity to the amino acid sequence set forth in SEQ ID NO:3 and the V_{L} region has at least 95% identity to the amino acid sequence set forth in SEQ ID NO:4;
the V_{H} region has at least 95% identity to the amino acid sequence set forth in SEQ ID NO:5 and the V_{L} region has at least 95% identity to the amino acid sequence set forth in SEQ ID NO:6; or
the V_{H} region has at least 95% identity to the amino acid sequence set forth in SEQ ID NO:7 and the V_{L} region has at least 95% identity to the amino acid sequence set forth in SEQ ID NO:8

6. The antibody of claim 4, comprising:
a V_{H} region having the sequence set forth in SEQ ID NO:1 and a V_{L} region having the sequence set forth in SEQ ID NO:2;
a V_{H} region having the sequence set forth in SEQ ID NO:3 and a V_{L} region having the sequence set forth in SEQ ID NO:4;
a V_{H} region having the sequence set forth in SEQ ID NO:5 and a V_{L} region having the sequence set forth in SEQ ID NO:6; or
a V_{H} region having the sequence set forth in SEQ ID NO:7 and a V_{L} region having the sequence set forth in SEQ ID NO:8.

7. A kit comprising a first IL-6Rα antibody of claim 4, coupled to a solid support.

8. The kit of claim 7, further comprising a second IL-6Rα antibody comprising at least one CDR that differs in sequence from the first IL-6Rα antibody.

9. The kit of claim 8, wherein the second antibody comprises:
(a)
HCDR1:
(SEQ ID NO: 10)
GFSFSGDYD
HCDR2:
(SEQ ID NO: 11)
TDSGFSGTT
HCDR3:
(SEQ ID NO: 12)
ARDFDSSGSYYWDL
LCDR1:
(SEQ ID NO: 13)
ESVSSNNR
LCDR2:
AAS
LCDR3:
(SEQ ID NO: 14)
AGYKGLYSDGRA;
(b)
HCDR1:
(SEQ ID NO: 15)
GFSFISYHYY
HCDR2:
(SEQ ID NO: 16)
IDAISSGST
HCDR3:
(SEQ ID NO: 17)
ARAPYYTYDGVYYALTL
LCDR1:
(SEQ ID NO: 18)
ENIYNF
LCDR2:
RAS
LCDR3:
(SEQ ID NO: 19)
QRNAYSSWA;
(c)
HCDR1:
(SEQ ID NO: 20)
GFSFSSGYY
HCDR2:
(SEQ ID NO: 21)
IYTGSDTT
HCDR3:
(SEQ ID NO: 22)
ARDLGSRGNL
LCDR1:
(SEQ ID NO: 23)
ENIYSF
LCDR2:
RAS
LCDR3:
(SEQ ID NO: 19)
QRNAYSSWA;
or
(d)
HCDR1:
(SEQ ID NO: 24)
GFSFSSNYW
HCDR2:
(SEQ ID NO: 25)
IYLFSVGNT
HCDR3:
(SEQ ID NO: 26)
ARAPYYLDGARAYYAFNL
LCDR1:
(SEQ ID NO: 27)
QSIGSD
LCDR2:
FAS
LCDR3:
(SEQ ID NO: 28)
AGGYNSADIFA.

10. The kit of claim 7, wherein the first antibody is coupled to a magnetic bead and the second antibody is labeled with a detectable label.

11. A method of detecting an IL-6Rα polypeptide comprising incubating a sample with a first IL-6Rα antibody of claim 4, wherein the antibody is labeled with a detectable label.

12. The method of claim 11, further comprising incubating the sample with a second IL-6Rα antibody comprising at least one CDR that differs in sequence from the first IL-6Rα antibody, wherein the antibody is coupled to a solid support.

13. The method of claim 12, wherein the second antibody comprises:
(a)
HCDR1:
(SEQ ID NO: 10)
GFSFSGDYD
HCDR2:
(SEQ ID NO: 11)
TDSGFSGTT
HCDR3:
(SEQ ID NO: 12)
ARDFDSSGSYYWDL
LCDR1:
(SEQ ID NO: 13)
ESVSSNNR
LCDR2:
AAS
LCDR3:
(SEQ ID NO: 14)
AGYKGLYSDGRA;
(b)
HCDR1:
(SEQ ID NO: 15)
GFSFISYHYY
HCDR2:
(SEQ ID NO: 16)
IDAISSGST
HCDR3:
(SEQ ID NO: 17)
ARAPYYTYDGVYYALTL
LCDR1:
(SEQ ID NO: 18)
ENIYNF
LCDR2:
RAS
LCDR3:
(SEQ ID NO: 19)
QRNAYSSWA;
(c)
HCDR1:
(SEQ ID NO: 20)
GFSFSSGYY
HCDR2:
(SEQ ID NO: 21)
IYTGSDTT
HCDR3:
(SEQ ID NO: 22)
ARDLGSRGNL
LCDR1:
(SEQ ID NO: 23)
ENIYSF
LCDR2:
RAS
LCDR3:
(SEQ ID NO: 19)
QRNAYSSWA;
or
(d)
HCDR1:
(SEQ ID NO: 24)
GFSFSSNYW
HCDR2:
(SEQ ID NO: 25)
IYLFSVGNT
HCDR3:
(SEQ ID NO: 26)
ARAPYYLDGARAYYAFNL
LCDR1:
(SEQ ID NO: 27)
QSIGSD
LCDR2:
FAS
LCDR3:
(SEQ ID NO: 28)
AGGYNSADIFA.

14. The method of claim 11 comprising detecting a signal from the detectable label.


SEQUENCE LISTING (29 sequences)

SEQ ID NO: 1 - protein, 140 aa, synthetic construct
METGLRWLLLVAVLKGVQCQSLEESGGGLVQPEGSLTLTCKASGFSFSGDYDMCWVRQAPGKGLEWIGCTDSGFSGTTYYASWAKGRFTISKTSSTTVTLQTTSLTAADTATYFCARDFDSSSGYYWDLWGPGTLVTVSS

SEQ ID NO: 2 - protein, 134 aa, synthetic construct
MDTRAPTQLLGLLLLWLPGATFAIVMTQTPSSKSVPVGDTVTINCQASESVSSNNRLAWYQQKPGQPPKLLIYAASTLTSGVPSRFKGSGSGTQFTLTISGVQCDDAATYYCAGYKGLYSDGRAFGGGTEVVVK

SEQ ID NO: 3 - protein, 145 aa, synthetic construct
METGLRWLLLVAVLKGVQCQEQLVESGGGLVQPEGSLTLTCKASGFSFISYHYYMCWVRQAPGKGLEWIACIDAISSGSTYYASWAKGRVTISKTSSTTVTLQMTSLTAADTATYFCARAPYYTYDYVGYALTLWGPGTLVTVSS

SEQ ID NO: 4 - protein, 130 aa, synthetic construct
MDTRAPTQLLGLLLLWLPGARCADIVMTQTPASVEAAVGGTVTINCQASENIYNFLTWYQQKPGQRPKLLIYRASTLASGVPSRFKGSGSGTDFTLTISDLECADAATYYCQRNAYSSWAFGGGTEVVVK

SEQ ID NO: 5 - protein, 135 aa, synthetic construct
METGLRWLLLVAVLKGVQCQSLEESGGDLVKPGASLTLACTASGFSFSSGYYMCWVRQAPGKGLEWIGCIYTGSDTTYYASWAKGRFTISETSSTAATLQMTSLTAADTATYFCARDLGSRGNLWGPGTLVTVSS

SEQ ID NO: 6 - protein, 130 aa, synthetic construct
MDTRAPTQLLGLLLLWLPGARCADIVMTQTPSSVSAAVGDTVTINCQASENIYSFLTWYQQKPGQRPKLLIYRASTLASGVPSRFKGSGSGTDFTLTISDLECADAATYYCQRNAYSSWAFGGGTEVVVK

SEQ ID NO: 7 - protein, 146 aa, synthetic construct
METGLRWLLLVAVLKGVQCQEHLEESGGGLVQPEGSLTLTCTASGFSFSSNYWICWVRQAPGKGLEWIACIYLFSVGNTFYASWAKDRFTISRSTSLNTVTLQMTSLTAADTATYFCARAPYYLDGYARAYAFNLWGPGTLVTVSS

SEQ ID NO: 8 - protein, 131 aa, synthetic construct
MDTRAPTQLLGLLLLWLPGARCAIDMTQTPASLSAAVGGTVAINCQASQSIGSDLAWYQQKPGQPPKLLIYFASKLASGVSSRFKGSGSGTQFTLTISDLECDDVATYYCAGGYNSADIFAFGGGTEVVVK

SEQ ID NO: 9 - protein, 365 aa, Homo sapiens [sequence omitted]

SEQ ID NO: 10 - protein, 9 aa, synthetic construct
GFSFSGDYD

SEQ ID NO: 11 - protein, 9 aa, synthetic construct
TDSGFSGTT

SEQ ID NO: 12 - protein, 14 aa, synthetic construct
ARDFDSSGSYYWDL

SEQ ID NO: 13 - protein, 8 aa, synthetic construct
ESVSSNNR

SEQ ID NO: 14 - protein, 12 aa, synthetic construct
AGYKGLYSDGRA

SEQ ID NO: 15 - protein, 10 aa, synthetic construct
GFSFISYHYY

SEQ ID NO: 16 - protein, 9 aa, synthetic construct
IDAISSGST

SEQ ID NO: 17 - protein, 17 aa, synthetic construct
ARAPYYTYDGVYYALTL

SEQ ID NO: 18 - protein, 6 aa, synthetic construct
ENIYNF

SEQ ID NO: 19 - protein, 9 aa, synthetic construct
QRNAYSSWA

SEQ ID NO: 20 - protein, 9 aa, synthetic construct
GFSFSSGYY

SEQ ID NO: 21 - protein, 8 aa, synthetic construct
IYTGSDTT

SEQ ID NO: 22 - protein, 10 aa, synthetic construct
ARDLGSRGNL

SEQ ID NO: 23 - protein, 6 aa, synthetic construct
ENIYSF

SEQ ID NO: 24 - protein, 9 aa, synthetic construct
GFSFSSNYW

SEQ ID NO: 25 - protein, 9 aa, synthetic construct
IYLFSVGNT

SEQ ID NO: 26 - protein, 18 aa, synthetic construct
ARAPYYLDGARAYYAFNL

SEQ ID NO: 27 - protein, 6 aa, synthetic construct
QSIGSD

SEQ ID NO: 28 - protein, 11 aa, synthetic construct
AGGYNSADIFA

SEQ ID NO: 29 - protein, 10 aa, synthetic construct
HHHHHHHHHH